(19) United States
(12) Patent Application Publication
(10) Pub. No.: US 2024/0138824 A1
(43) Pub. Date: May 2, 2024

(54) VASCULAR CLOSURE DEVICES AND METHODS

(71) Applicant: Arterica Inc., Santa Rosa, CA (US)

(72) Inventors: Brian HAUCK, Windsor, CA (US); Thomas LARZON, Lidingö (SE); Joseph W. HUMPHREY, Santa Rosa, CA (US); Robert G. WHIRLEY, Santa Rosa, CA (US); Robert L. CARTER, Petaluma, CA (US); Dion J. THUROW, Santa Rosa, CA (US)

(21) Appl. No.: 18/496,641

(22) Filed: Oct. 27, 2023

Related U.S. Application Data
(60) Provisional application No. 63/420,391, filed on Oct. 28, 2022.

(51) Int. Cl. A61B 17/00

(52) U.S. Cl. CPC A61B 17/0057; A61B 2017/00367; A61B 2017/00659

(57) ABSTRACT

Vascular closure assembly device and method embodiments may be used to provide hemostasis at vascular puncture site or the like. Such vascular puncture or access sites may be created during a variety of percutaneous or minimally invasive medical procedures.

### RELATED APPLICATIONS

This application claims priority under 35 U.S.C. section 119(e) from U.S. Provisional Patent Application No. 63/420,391 filed Oct. 28, 2022, by B. Hauck et al., titled “Large Bore Closure Devices and Methods”, which is incorporated by reference herein in its entirety.

### BACKGROUND

In many percutaneous procedures, a catheter is inserted into an access hole in a blood vessel, such as the femoral artery. Such percutaneous procedures may include minimally invasive cardiovascular procedures including, for example, balloon angioplasty procedures, atherectomy procedures, cardiovascular stent deployment, heart valve replacement, stent graft deployment, as well as others. During such procedures, a therapeutic catheter may be inserted, typically over a guidewire, directly into an artery, or the catheter may be inserted through a vascular introducer sheath. When the therapeutic procedure is complete, the physician generally removes the therapeutic catheter and then removes the introducer sheath from the vessel (if one was used). The physician then must prevent or limit the amount of blood that leaks through the vascular access hole in the wall of the affected vessel. Physicians currently use a number of methods to close the vascular access hole or otherwise limit bleeding post procedure from the access hole, such as localized external compression, suture-mediated closure devices, cut-down direct suture mediation, plugs, gels, foams and similar materials.

However, such closure procedures may be time consuming, and may consume a significant portion of the time of the procedure. In addition, some existing methods are associated with complications such as hematoma or thromboses. Still further, some of such procedures, particularly suture-mediated closure devices, are known to have high failure rates in the presence of common vascular diseases such as atherosclerosis and calcification. What has been needed are methods and devices that may be used to efficiently and conveniently close a vascular access hole after a procedure has been completed.

### SUMMARY

Some embodiments of a vascular closure assembly may include an actuator assembly that has a chassis portion and an elongate housing with a proximal end thereof secured to a distal end of the chassis portion, a distal end extending away from the chassis portion, a distal section, and a plurality of anchor deployer lumens. Each anchor deployer lumen may extend axially or in any other suitable path along the elongate housing and terminate distally at a distal port disposed in the distal section of the elongate housing. The actuator assembly may further include a plurality of anchor deployers, each anchor deployer being slidably disposed within a respective anchor deployer lumen of the elongate housing. Each anchor deployer may include a deployment rod which has an elongate resilient configuration and a pre-shaped distal section that assumes a curved profile while in a relaxed state. The pre-shaped distal section may also have a straightened profile while in a constrained state within the respective anchor deployer lumen and may be configured to extend from the respective distal port of the anchor deployer lumen along a curved path as an extended portion of the pre-shaped distal section relaxes and assumes the curved profile. The anchor deployer may also include an anchor which may be removably secured to the distal end of the deployment rod and which may be configured to resist proximal retraction within tissue. A respective filament may be secured to each anchor of the anchor deployers.

Some embodiments of a vascular closure assembly may have an actuator assembly including a chassis portion and a plurality of anchor deployers with each anchor deployer including a deployment rod, an anchor which is removably secured to a distal end of the deployment rod, and a filament secured to each anchor. The actuator assembly may further include an elongate housing having a proximal end secured to a distal end of the chassis portion, a distal end, and an inner lumen extending along the elongate housing to the distal end of the elongate housing. The actuator assembly may also include a plurality of anchor deployer lumens configured to be slidably disposed about a respective anchor deployer with each anchor deployer lumen extending along the elongate housing and terminating distally at a distal port disposed in the distal section of the elongate housing. A plurality of filament retainers may be disposed on an outside surface of the elongate housing proximally of the distal ports of the anchor deployer lumens. In some instances, each filament retainer may be configured to releasably secure a section of a respective filament.

Some embodiments of a vascular closure assembly may include an inner catheter assembly having an elongate shaft with a proximal end, a distal end, a distal section, an axial length and a guidewire lumen that extends proximally from a distal port at the distal end of the elongate shaft to a proximal port disposed at the distal section. The vascular closure assembly may also include an actuator assembly having a chassis portion and a plurality of anchor deployers with each anchor deployer including a deployment rod, an anchor which is removably secured to a distal end of the deployment rod, and a filament secured to each anchor. The actuator assembly may also have an elongate housing that includes a proximal end secured to a distal end of the chassis portion, a distal end, and an inner lumen extending along the elongate housing to the distal end of the elongate housing, the inner lumen having an inner surface contour which is configured to be slidably disposed over an outer surface of the elongate shaft. The elongate housing may further include a plurality of anchor deployer lumens configured to be slidably disposed about a respective anchor deployer, each anchor deployer lumen extending along the elongate housing and terminating distally at a distal port disposed in the distal section of the elongate housing. The elongate housing may include a guidewire relief slot which is disposed in the inner lumen through a wall portion thereof and which extends proximally from the distal end of the inner lumen to a proximal end of the guidewire relief slot. Such a guidewire relief slot may be configured to accommodate a guidewire extending outwardly from the proximal port of the guidewire lumen of the elongate shaft. The elongate housing may also optionally have a guidewire retention clip which extends outwardly from an outer surface of the elongate housing and which is disposed proximally of the proximal end of the guidewire relief slot.

Some embodiments of a vascular closure assembly may include an actuator assembly having a chassis with a distal end and a proximal end, a plunger which is proximally translatable with respect to the chassis over a retraction length starting from a distal position, and a tensioner which has a first end secured to the chassis, a second end releasably secured to the plunger and which is configured to continuously apply proximally oriented tension to the plunger with respect to the chassis over the retraction length. The actuator assembly may also include a trigger latch that releasably secures the plunger in the distal position in opposition to the tensioner and a platen that is distally translatable with respect to the plunger from a proximal cocked position over a deployment length to a distal position that actuates the trigger latch and releases the plunger allowing proximal translation of the plunger over the retraction length. The actuator assembly may also include a compression spring which has a first end operatively coupled to the plunger, a second end operatively coupled to the platen and which is configured to apply a distally oriented force to the platen from the proximal cocked position of the platen over the deployment length to the distal position of the platen. A platen latch may be operatively coupled to the chassis with a configuration allowing actuation of the platen latch but preventing distal translation of the platen latch with respect to the chassis. The platen latch may include a platen catch that is operatively coupled to the platen so as to releasably secure the platen in the proximal cocked position. An actuation button may be operatively coupled to the platen latch and configured to actuate the platen latch to disengage the platen catch from the platen. The actuator assembly may also have an elongate housing with a proximal end thereof secured to a distal end of the chassis and a plurality of anchor deployers with each anchor deployer being slidably disposed within a respective anchor deployer lumen of the elongate housing. Each anchor deployer may include a deployment rod having an elongate resilient configuration which is operatively coupled to the platen such that distal translation of the platen results in distal translation of the deployment rod and an anchor which is removably secured to the distal end of the deployment rod.

Some embodiments of a method of actuating an actuator assembly of a vascular closure assembly may include actuating a platen latch of the actuator assembly with an actuation button operatively coupled to a chassis thereby releasing a compression spring, which is operatively coupled between a plunger and a platen, from a compressed state. Thereafter, translating the platen and deployment rods operatively secured thereto in a distal direction relative to the plunger and chassis under a distal force generated by the released compression spring then actuating a trigger latch which releasably secures the plunger in a distal position with the platen as it translates distally, thereby releasing the plunger from the fixed distal position. Lastly, the method may include translating the plunger, platen and deployment rods secured to the platen in a proximal direction under a proximal force generated by a tensioner which is secured to the chassis and releasably secured to the plunger.

Some embodiments of a vascular closure assembly may include a chassis and an elongate housing having a proximal end secured to a distal end of the chassis. A plurality of anchor deployers may be configured to extend from a distal section of the elongate housing with each anchor deployer including an anchor and a filament secured to the anchor. The elongate housing may include a filament lock assembly having filament tube, a filament lock having an inner lumen, the inner lumen being disposed over an outer surface of a distal section of the filament tube, and a fairlead having an inner lumen which is disposed over the filament tube axially adjacent the filament lock. A polymer load transfer bushing may further be disposed between the fairlead and the filament lock.

Certain embodiments are described further in the following description, examples, claims and drawings. These features of embodiments will become more apparent from the following detailed description when taken in conjunction with the accompanying exemplary drawings.

### BRIEF DESCRIPTION OF THE DRAWINGS

FIG. 1 shows a perspective view of a vascular closure assembly embodiment including an actuator assembly that includes a chassis handle portion and an elongate housing extending therefrom as well as an inner catheter assembly positioner disposed within an inner lumen of the actuator assembly.

FIG. 2 shows the vascular closure assembly embodiment of FIG. 1 with a positioner lever embodiment of the inner catheter assembly in a deployed configuration.

FIG. 3 is a perspective view of the vascular closure assembly of FIG. 1 with half of the outer shell of the chassis portion not shown for purposes of illustration.

FIG. 4 is a perspective view of a distal portion of the inner catheter assembly of the vascular closure assembly of FIG. 1 with an inflatable balloon of the inner catheter assembly shown in a deflated configuration and a foot extension of the inner catheter assembly shown in a retracted configuration in a pre-deployment state.

FIG. 4A is a schematic view in elevation of the distal section of the inner catheter assembly of FIG. 4.

FIG. 5 is a perspective view of the distal portion of the inner catheter assembly of the vascular closure assembly of FIG. 1 with the inflatable balloon of the inner catheter assembly shown in an inflated state and the foot extension of the inner catheter assembly shown in an axially extended deployed state.

FIG. 5A is a schematic view in elevation of the distal section of the inner catheter assembly of FIG. 5.

FIG. 6 is a perspective view of the distal portion of the inner catheter assembly of the vascular closure assembly of FIG. 1 with the inflatable balloon in a post-deployed state and wherein the blood has been evacuated out of the inflatable balloon.

FIG. 7 is a perspective view of a one-way valve embodiment of the inner catheter assembly shown in an open configuration.

FIG. 8. is a perspective view of the one-way valve embodiment of FIG. 7 shown in a closed state.

FIG. 9 is a perspective view in partial section illustrating guidewire routing in an elongate housing embodiment of the vascular closure assembly and the distal section of the inner catheter assembly.

FIG. 10 is an elevation view in partial longitudinal section of a proximal section of the chassis portion of the vascular closure assembly of FIG. 1 and the associated proximal portion of the inner catheter assembly illustrating detent latch embodiments of the inner catheter assembly.

FIG. 11A shows a perspective view of the proximal section of the chassis in longitudinal section with the inner catheter assembly embodiment engaged with a detent and spring embodiments pushing an interlock to definitively lock the inner catheter assembly embodiment in a fixed axial location relative to the chassis portion.

FIG. 11B shows the proximal section of the chassis of FIG. 11A illustrating depression of the interlock of the embodiment of FIG. 11A to allow the inner catheter assembly embodiment to move out of the interlocked position and translate axially with respect to the chassis portion.

FIG. 12 is a perspective view in partial section of a distal section nosetip of the elongate housing embodiment of the actuator assembly of the vascular closure assembly of FIG. 1.

FIG. 12A is an end view of the filament lock bushing embodiment of FIG. 12.

FIG. 13 is an elevation view of the vascular closure assembly of FIG. 1 with half of the outer shell of the chassis portion not shown for purposes of illustration and with the vascular closure assembly disposed in a loaded configuration ready for deployment.

FIG. 13A is an enlarged view showing the engagement of a trigger latch embodiment and plunger embodiment of FIG. 13.

FIG. 14 is an elevation view of the vascular closure assembly of FIG. 13 with the vascular closure assembly disposed in a state of initial deployment with the deployment rod actuator compression spring released wherein the anchor deployers (not shown) would be disposed in a distally extended and deployed state as shown in FIG. 17.

FIG. 15 is an elevation view of the vascular closure assembly of FIG. 14 with the vascular closure assembly disposed in a state of initial filament retraction and withdrawal of the deployment rods of the anchor deployer embodiments.

FIG. 16 is a perspective view of the vascular closure assembly of FIG. 15 during secondary tensioning of the filaments using a knob and threaded barrel engaged with a tubular plunger and wherein deployment of filament locks (not shown) onto the filaments using a filament lock assembly would also be taking place as shown in FIG. 19.

FIG. 17 is a perspective view of the distal section of the elongate housing of the vascular closure assembly during deployment of the deployment rods and associated anchors of the anchor deployers of the device embodiment.

FIG. 18 is a perspective view of the distal nosetip section of the elongate housing of the actuator assembly of FIG. 17 after proximal retraction and withdrawal of the deployment rods of the anchor deployers post deployment.

FIG. 19 is a perspective view of the filaments, filament locks and anchors of the vascular closure assembly of FIG. 18 after the filaments have been properly tensioned and the filament locks deployed onto the filaments.

FIG. 20 is an elevation view of an elongate housing embodiment without any additional structure of a vascular closure assembly shown for purposes of illustration.

FIG. 21 is a transverse cross section of the elongate housing embodiment of FIG. 20 taken along lines 21-21 of FIG. 20.

FIG. 21A is an enlarged transverse cross section of a filament retainer embodiment of the elongate housing embodiment of FIG. 21.

FIG. 22 is a perspective view of the elongate housing embodiment of FIG. 20 with a plurality of deployment rods disposed therein in an extended deployed state with proximal ends of the deployment rods shown secured to a platen embodiment.

FIG. 23 is a perspective view of the plurality of deployment rods and platen of FIG. 22.

FIG. 24 is an end view of the elongate housing embodiment, deployment rods and platen of FIG. 22.

FIG. 25 is an end view of the deployment rods and platen of FIG. 24.

FIG. 26 is a top view of the cranial deployment rod embodiments of FIG. 25 lying in the plane of the page.

FIG. 27 is a side view of the caudal deployment rod embodiments of FIG. 25 with one of the pre-shaped distal sections thereof lying in the plane of the page.

FIG. 28 is a perspective view of a vascular closure assembly embodiment.

FIG. 29 is an elevation view in partial section of the vascular closure assembly embodiment of FIG. 28.

FIGS. 30-32 show schematic representations of a nosetip of an elongate housing embodiment and chassis embodiment of a vascular closure assembly embodiment in partial section and with multiple break aways during actuation/retraction of a filament tube embodiment thereof.

The drawings are intended to illustrate certain exemplary embodiments and are not limiting. For clarity and ease of illustration, the drawings may not be made to scale, and in some instances, various aspects may be shown exaggerated or enlarged to facilitate an understanding of particular embodiments.

### DETAILED DESCRIPTION

Embodiments of the devices, which may include vascular closure devices or assemblies, discussed herein may be used for percutaneous closure of an access hole into a body lumen, such as an artery including the common femoral artery. Embodiments of vascular closure assemblies may work by using extension wires, sometimes referred to herein as deployment rods, to place a plurality of anchors, such as three, four or more anchors through a tissue layer, such as a fascia layer, in a pattern circumferentially disposed around a passage in the tissue layer disposed adjacent the access hole in the vessel of an access site. A filament such as a suture or any other suitable filament embodiment may be connected or otherwise secured to each anchor, with the filaments extending from the respective anchors and entering a distal nosetip section of an elongate housing of the device through a distal port of a filament tube at a distal end of the nosetip of the elongate housing. The filaments may then extend proximally through an inner lumen of this filament tube and may ultimately be connected or otherwise secured either directly or indirectly to a tensioner such as spring or the like.

For some embodiments, during deployment, these filaments may be proximally tensioned from respective anchored positions at their distal ends to a common point such as the distal port of the filament tube. Such tension thereby pulls the tissue layer together to close the passage in the tissue layer and concurrently isolate and prevent blood leakage from the access hole in the patient's vessel. As mentioned above, for some embodiments, the access hole in the patient's vessel may be disposed below and adjacent the associated passage in the tissue layer. A filament lock embodiment may then be deployed from the nosetip of the elongate housing onto the filaments, and the filaments may then be cut by an internal mechanism in a chassis portion handle or by any other suitable mechanism.

Embodiments of similar systems and methods are discussed in U.S. Pat. No. 11,179,145, filed Nov. 14, 2018, by T. Larzon et al. and titled “Collapsible Tube for Hemostasis,” U.S. Patent Publication No. 2019/0142403, filed Nov. 14, 2018, by H. Nyman et al. and titled “Tissue Closure Device,” U.S. Pat. No. 10,639,020, filed Sep. 27, 2016, by T. Larson et al. and titled “Vascular Closure Device,” U.S. Patent Publication No. 2020/0129164 filed Oct. 23, 2019 by T. Larzon et al. and titled “Self-Expanding Hemostatic Devices and Methods for Fascia and Vessel Passages,” and U.S. Patent Publication No. 2021/0145421, filed Nov. 18, 2020 by B. Hauck et al. and titled “Vascular Closure Devices and Methods,” each of which is incorporated by reference herein in its entirety. Any suitable feature, dimension or material of the embodiments of these incorporated references may be used in any of the appropriate embodiments discussed herein.

In some cases, vascular closure assembly embodiments may include two major components consisting of an actuator assembly, and a positioner, also referred to herein as an inner catheter assembly. The actuator assembly may include a handle, also referred to herein as a chassis or chassis portion, and an elongate housing that extends distally from the chassis. The inner catheter assembly may include a small lumen extending a length thereof to provide an indication that the distal tip of the inner catheter assembly is disposed within a lumen of a target vessel, such as an artery which may include the common femoral artery. Inner catheter assembly embodiments may also include a foot extension for positioning against an anterior wall of the vessel from within the interior lumen of the vessel, and an inflatable balloon (which may be inflated by blood pressure from within the artery) for maintaining hemostasis during the procedure. The elongate housing may extend from the chassis portion and may be used to at least partially house and facilitate the deployment of a plurality of anchor deployer embodiments, such as three, four or more anchor deployers. In some cases, each anchor deployer may include an anchor, with a respective filament, such as a suture, attached or otherwise secured to the anchor.

For some deployment method embodiments, the anchors may be implanted at locations circumferentially disposed around a passage through a tissue layer, such as a fascia layer, disposed adjacent an access hole in the vessel by deployment rods. The deployment rods may be distally advanced or otherwise actuated by release of a spring, such as a compression spring in some cases. The compression spring may be released or otherwise deployed by a button on the chassis portion in some cases. An internal mechanism in the chassis portion may be used to control another spring (which may include a constant force-type tension spring in some instances) to automatically retract the deployment rods once the anchors are advanced through the tissue layer. This constant force spring may also be used for applying tension to the filaments to close the passage in the tissue layer. Filament lock embodiments may be deployed onto the tensioned filaments to hold the filaments in place and in fixed relation to each other once a filament connection to the chassis portion components has been cut or otherwise disconnected.

Some vascular closure assembly embodiments may include good intuitive ergonomics for ease-of-use. Some such vascular closure assembly device embodiments may also include a reduced or otherwise low profile nosetip/distal portion of the elongate housing which may be configured to allow for insertion of the nosetip directly upon sheath removal during a deployment procedure without any requirement for preparation (e.g., manual enlargement) of a tissue tract such as the passage in the tissue layer, access hole in the vessel being used or dermal tissue or any other associated tissue disposed above the tissue layer. The low profile of the nosetip may also enable an entire closure procedure to be accomplished with a longitudinal axis of the elongate housing of the device being disposed at an angle of about 45 degrees with respect to a longitudinal axis of the subject blood vessel of the patient (a natural guidewire entry angle). As such, there may be no need to elevate or otherwise alter the orientation of the vascular closure assembly device to alter the angle of the device with respect to the subject vessel during a deployment procedure thus increasing the ease of use for the operator.

Some vascular closure assembly embodiments may include pre-shaped deployment rods in order to facilitate a desired distribution of the anchors about the passage in the tissue layer. Some such deployment rod embodiments may be constructed of a resilient shape-set material such as nitinol, including superelastic nitinol, or the like. Such pre-curved geometries of a distal section of the deployment rods may allow a desired anchor deployment pattern around the passage while also maintaining the longitudinal axis of the vascular closure assembly at the natural 45 degree angle, or any other suitable angle, with respect to the longitudinal axis of the targeted blood vessel. Some such embodiments may include two general types of deployment rods depending on the circumferential position with respect to the longitudinal axis of the nosetip of the elongate housing. In some cases, the two types of deployment rods may include cranial deployment rods and caudal deployment rods. For some embodiments, the cranial deployment rods may have an optimized geometry that is different from the geometry of the caudal deployment rods, as may be naturally required by the angle of the handle or chassis portion relative to the patient's anatomy during deployment. These shaped deployment rods, along with the nosetip configuration, may, in some instances, allow the same device to be used on either the right groin or left groin of the patient for some embodiments.

For some of the vascular closure assembly embodiments discussed herein, the deployment rods may be spring-driven for deployment and tissue penetration by the anchors as discussed above. Spring-driven deployment may eliminate variability in anchor deployer performance due to varying operator input or the like in some cases. Deployment of the anchors for some device embodiments may be accomplished by simply pressing an actuator button on the chassis of the device. In some instances, the actuator button may be located on top of the chassis of the vascular closure assembly so that it can be conveniently reached from either side of the device, as some operators prefer to reach across the table for a contralateral deployment while other operators prefer to work from the contralateral side of the table for a contralateral deployment.

For some embodiments, features including a single control for foot extension actuation and actuation of the balloon inflation valve may be included which combines both functions, thereby simplifying the user experience. Detents may be provided on the inner catheter assembly, with matching engagement features on the chassis handle, to make it easier for the operator to slide the chassis down the elongate shaft of the inner catheter assembly into a correct relative axial location for deployment. Detents may also be provided to guide the operator with regard to how far to retract the inner catheter assembly after the filaments have been tensioned. This feature may, in some instances, eliminate the need for the operator to visually reference alignment marks on the inner catheter assembly when translating the chassis up or down along the inner catheter assembly during the deployment sequence.

For some vascular closure assembly embodiments, filament lock deployment may be carried out with the filament tensioning knob, rather than using a separate control on the chassis handle. For such embodiments, the operator may simply turn the tensioning knob a fixed number of turns (e.g., four) to tension the filaments, then retract the inner catheter assembly, then continue turning the same tensioning knob until it stops thereby deploying the filament lock. This may simplify device operation for the user in some circumstances. An interlock may be integrated into the filament tensioning knob mechanism so that the filament tensioning knob stops after a fixed number (e.g., four) turns, so that the operator does not inadvertently turn the knob too far prior to withdrawing the inner catheter assembly.

On the distal end of the elongate shaft of the inner catheter assembly, the guidewire may exit the elongate shaft just behind or proximal of the nosecone of the inner catheter assembly (“rapid exchange” style) and pass through a guide hole in a tab on the posterior aspect of the nosetip of the elongate housing. This configuration may allow the profile of the inner catheter assembly to be significantly reduced, which may be beneficial for allowing the overall outer profile of the nosetip (through which the inner catheter assembly passes) to be reduced.

The operation of some device embodiments discussed herein to close an access hole in a vessel of a patient may begin once an endovascular procedure is complete and while a guidewire is in place disposed through the access hole and within the patient's vessel lumen and an associated passage through the tissue layer disposed over the vessel. The actuator assembly with the inner catheter assembly may first be loaded over the guidewire, and then advanced through the passage and into the access hole (while hemostasis is maintained via manual compression) until a visible blood return appears on the proximal end of the inner catheter assembly. A lever may then be raised or otherwise actuated to deploy the foot extension and allow filling the inflatable balloon, and the actuator assembly and inner catheter assembly may be pulled in a proximal direction until the foot extension engages with an inner surface of an anterior wall of the patient's vessel adjacent the access hole in the vessel. The hemostasis inflatable balloon soon fills expanding outwardly against the perimeter of the access hole, thereby providing temporary bleeding control at the access site. Manual compression may now be released.

The actuator assembly may then be slid distally over the inner catheter assembly until it engages a detent, thereby positioning the nosetip at the end of the elongate housing at a correct distance from the vessel (and the tissue layer which is disposed over and adjacent the vessel). Next, the button on the chassis is pressed to deploy the anchor deployers and associated anchors into and through the tissue layer, which may include a fascia layer. Filament tension may then be applied by rotating the large knob at the proximal end of the chassis. The foot extension is then retracted and the balloon inflation valve closed by lowering the lever, and the inner catheter assembly is then withdrawn proximally into the inner lumen of the elongate housing, thereby allowing filament tension to fully close the access hole in the fascia layer. Finally, the filament lock is deployed by the filament lock assembly by continued rotation of the suture tensioning knob until it stops, and the filaments are cut by pulling a tab/trigger on the bottom of the chassis. The entire vascular closure assembly may now be slid proximally off the guidewire, the guidewire withdrawn from the patient's vessel and the skin wound closed in standard fashion.

Some vascular closure assembly embodiments may include one or more or any combination of the following features. In some cases, a distal section of the deployment rods may have a preset shape that enables a desired tissue layer penetration pattern and angle of incidence with the longitudinal axis of the chassis and elongate housing of the device positioned at an angle of about 45 degrees with respect to an axis of the vessel of the patient. In some cases, the 45 degree deployment angle may be representative of a typical and natural angle for guidewire entry and associated interventional devices into the lumen of the patient's vessel. In some cases, a single pushbutton-activated mechanism that uses stored energy (e.g., a compressed spring) to advance the deployment rods in a forward distal direction by a set distance into a tissue layer such as a fascia layer may be used. Thereafter, a plunger may be deployed which allows a second spring to retract the deployment rods and/or apply tension to the filaments with no further input from the operator.

For some embodiments, a rotational knob on the rear (proximal end) of the chassis may be configured to allow a slow, progressive tightening of the filaments until a prescribed predetermined tension is reached, after which the filament tension is governed by a constant force spring and further turns of the knob do not affect filament tension. For some embodiments, subsequent turns of the knob deploy the filament lock. Detent features on the chassis and the inner catheter assembly may be configured to provide clear, definitive feedback to the operator when the components of vascular closure assembly are in the correct position for deploying the anchors, and subsequently when the inner catheter assembly is fully retracted prior to filament lock deployment. In some cases, a single lever on the proximal end of the elongate shaft of the inner catheter assembly may be configured to both actuate the foot extension and simultaneously actuate the balloon inflation valve that allows hemodynamic pressure to fill the inflatable balloon. A simple lever-activated filament cutter may be configured to allow the operator to cut the filaments easily just prior to withdrawal of the vascular closure assembly from the patient.

For some embodiments, the anchor configuration and filament-anchor connection may be the same as or similar to those discussed in U.S. Patent Publication No. 2021/0145421, filed Nov. 18, 2020 by B. Hauck et al. and titled “Vascular Closure Devices and Methods,” which is incorporated by reference herein in its entirety. In addition, filament lock embodiments discussed herein may be the same as or similar to those discussed in this same publication. Some vascular closure assembly embodiments may also include one or more or any combination of the following features. For example, during deployment, instead of 4 turns, stop and remove the inner catheter assembly, and then finish turning knob to stop—complete all turns to stop in one sequence and remove inner catheter assembly after all turns. In some cases, the twist knob does not deploy the filament lock for such embodiments. In some instances, the filament cutting lever may be configured to both deploy the filament lock and cut the filaments allowing the chassis to be removed.

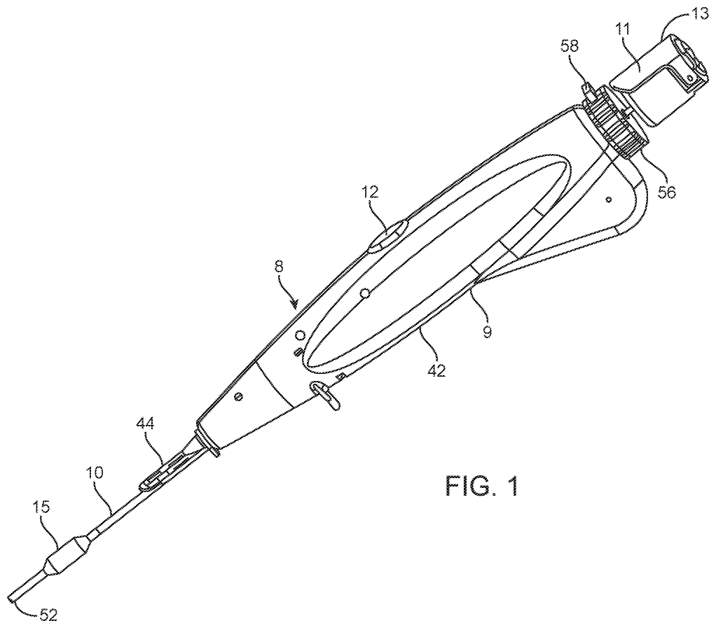

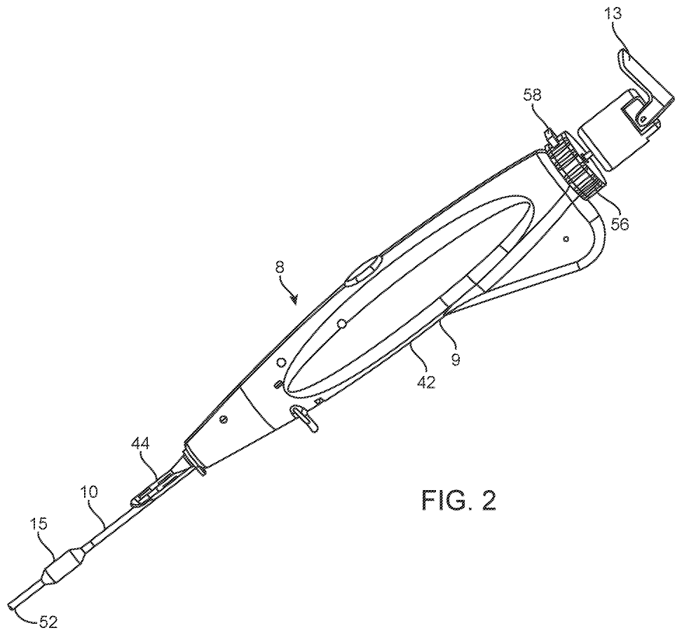

Referring to FIGS. 1-6, an embodiment of an actuator assembly 9 and associated inner catheter assembly 10 of a vascular closure assembly embodiment 8 are shown. The inner catheter assembly 10 may be a thin cylindrical device that traverses completely through the chassis 42 and elongate housing 44 of the actuator assembly 9. The inner catheter assembly 10 may also have a larger round hub 11 and lever 13 at the proximal end of the chassis 42 and thin tube such as an elongate shaft 46 with an inflatable balloon 15, foot extension 14, balloon inflation valve 48, and guidewire tracking distal section nosecone 24. The main device deployment button 12 is located on the top of the chassis 42 for easy ambidextrous use in either the right or left hand of the operator. FIG. 2 shows the positioner lever 13 in a deployed state and this deploys the foot extension 14 that is located inside the inflatable balloon 15 at the distal aspect of the inner catheter assembly 10. The lever 13 is also configured to open the balloon inflation valve 48 distal to the foot extension 14 that allows blood to enter and fill the inflatable balloon 15 to provide hemostasis during the procedure.

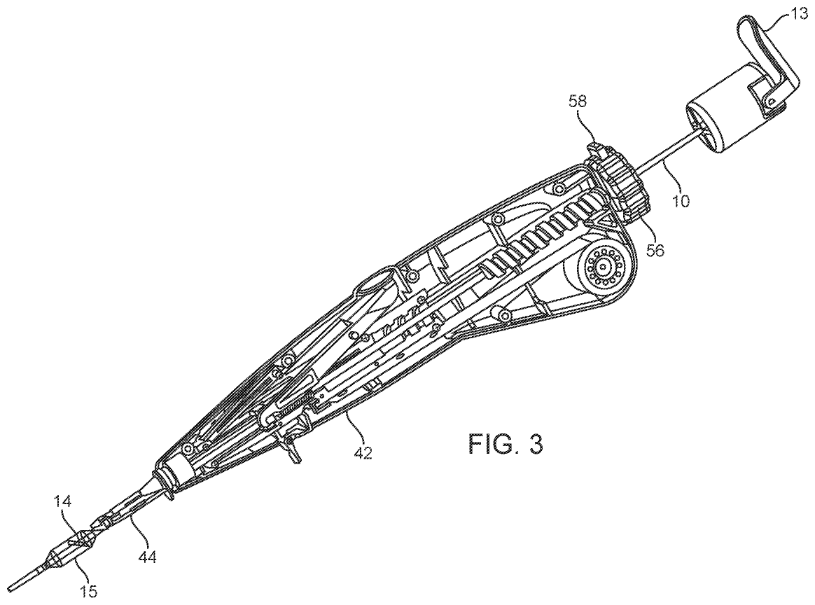

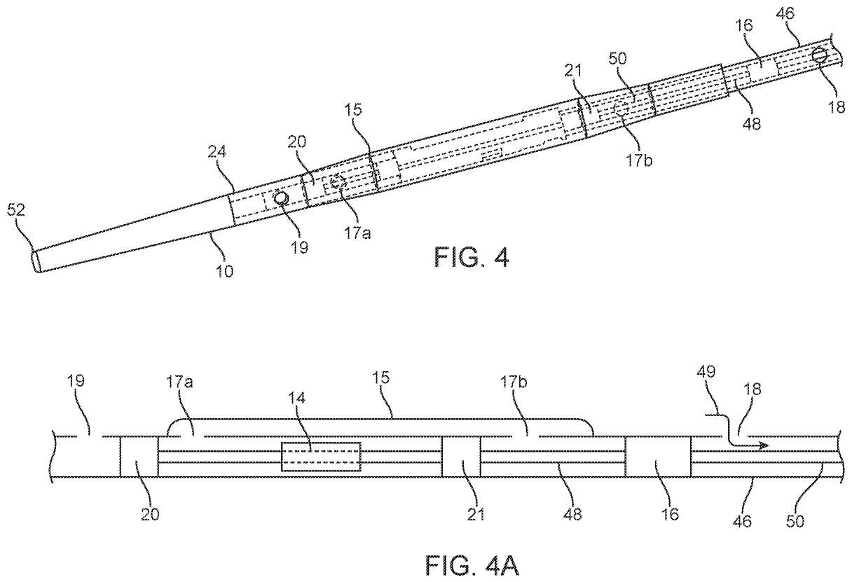

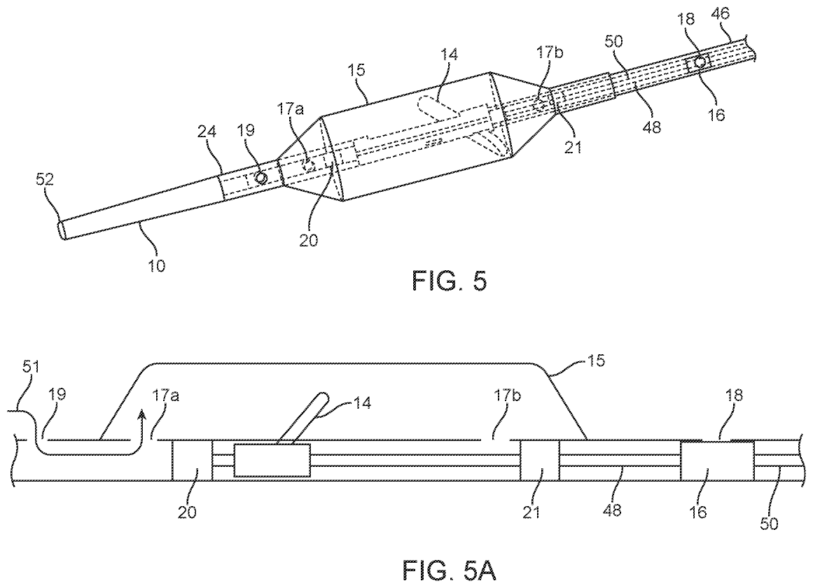

FIG. 3 is a transverse section view through an embodiment of the chassis 42 with the positioner lever 13 lifted which results in the foot extension 14 being deployed. The lever 13 may be operatively coupled to an actuation wire 50, a distal section of which is shown in FIGS. 4-6 in conjunction with the structure and function of the balloon inflation valve 48. This actuation wire 50 may be configured to actuate the foot extension 14 and translate two plugs 20, 21 along with a one-way valve 16. FIGS. 4 and 4A show an embodiment of the tip 24 of the inner catheter assembly 10 in a pre-deployed state with the positioner lever 13 in a non-pulled state as shown in FIG. 1. The inflatable balloon 15 is deflated and the plugs 20, 21 and one-way valve 16 are positioned to allow blood to enter the blood return hole 18 and flow proximally through the inner catheter assembly 10 as indicated by arrow 49 to provide an indication to the operator that the distal end 52 of the inner catheter assembly 10 is within the inner lumen of the patient's vessel also indicating that the foot extension 14 can be deployed. There are two holes 17a, 17b in the flexible lumen of the elongate shaft 46 contained within the inflatable balloon 15. There is an additional hole 19 distal to the inflatable balloon 15 that allows inflation of the inflatable balloon 15 when the positioner lever 13 is lifted/actuated resulting in the plugs 20, 21 and foot extension 14 translating proximally to the fill position as shown in FIGS. 5 and 5A.

FIGS. 5 and 5A show the embodiment of the tip 24 of the inner catheter assembly 10 in a deployed state with the positioner lever 13 actuated/lifted as shown in FIGS. 2 and 3. The foot extension 14 is extended and the distal plug 20 and proximal plug 21 have translated across the two holes 17a, 17b contained within the inflatable balloon 15 to positions proximal of those holes 17a, 17b, respectively. With the inner catheter assembly 10 in the deployed state, blood can enter the distal hole 19 as indicated by arrow 51 and flow through the lumen and fill the inflatable balloon 15 via the internal holes 17a, 17b. Blood is prevented from flowing out the inflatable balloon 15 and down the blood return lumen due to the proximal plug 21 blocking the flow path.

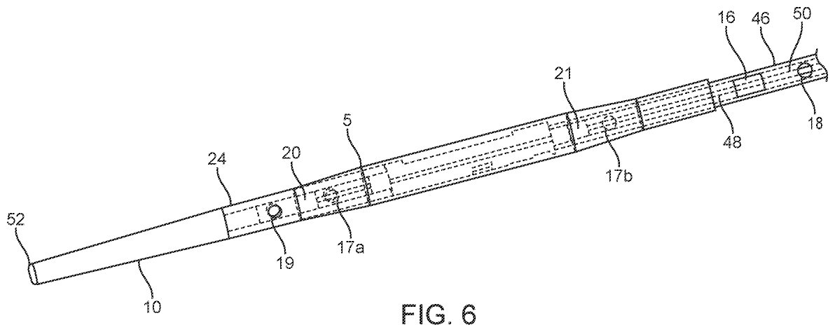

FIG. 6 shows the inner catheter assembly embodiment 10 with the foot extension 14 retracted after the deployment has been performed. The plugs 20,21 have transitioned back across the internal holes 17a, 17b, to positions distal of holes 17a, 17b, respectively as shown in FIGS. 4 and 4A. Arterial blood or any other source of pressurized fluid within a vessel can no longer fill the inflatable balloon 15 due to the distal plug 20 blocking the flow that comes in through distal hole 19. Blood that was contained within the inflatable balloon 15 during the actuated state shown in FIGS. 5 and 5A can now exit through the proximal hole 17b and flow through the one-way valve 16 and out the blood return lumen. This configuration may be configured to implement the feature that once blood flows into the inflatable balloon 15 it is never returned to the patient's vessel, thereby eliminating any potential risk of thrombosis from the stagnant blood in the inflatable balloon 15 being returned to the patient's body.

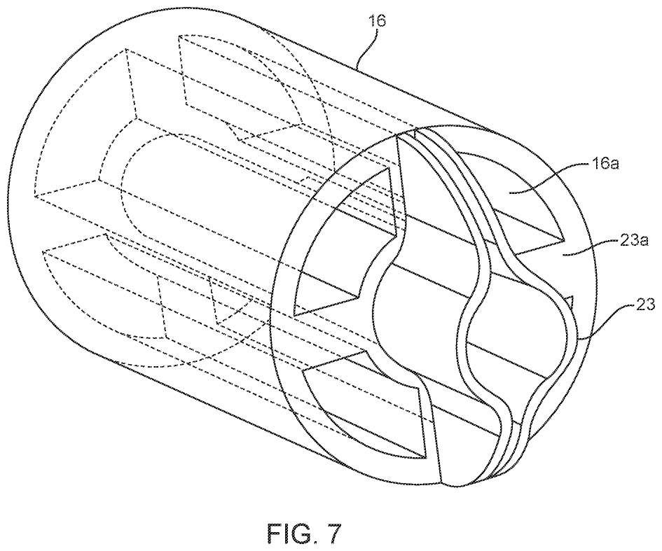

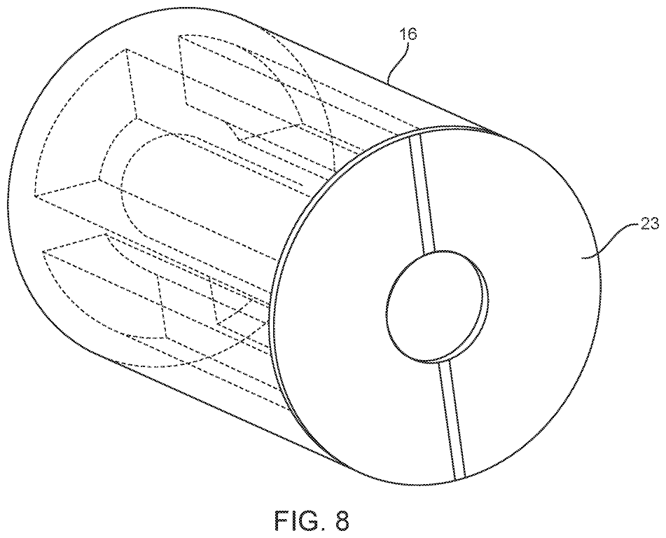

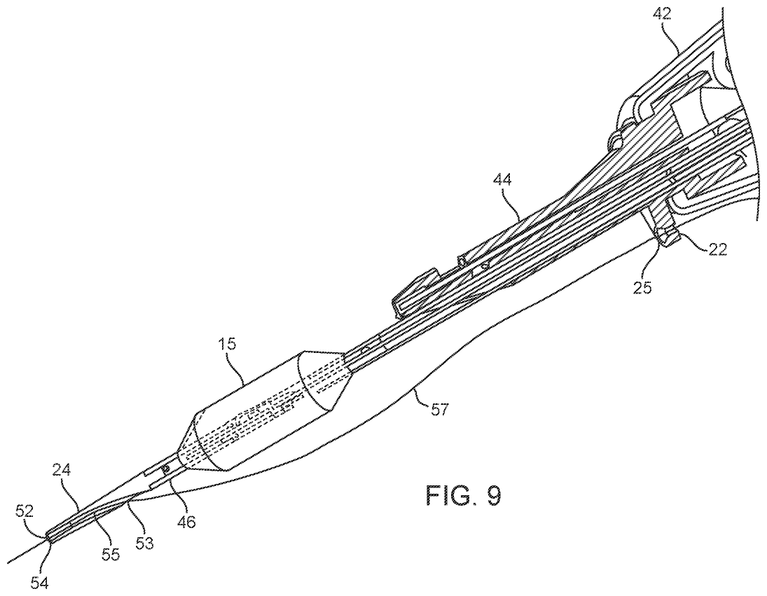

FIGS. 7 and 8 show the one-way valve embodiment 16 with the flapper 23 in the open and closed positions, respectively. FIG. 7 shows the flapper 23 in an open position away from the proximal sealing surface 23a with flow channels 16a open to allow a flow of blood therethrough in a proximal direction past the flapper 23. FIG. 8 shows the flapper 23 pressed against the proximal sealing surface 23a with the flow channels 16a closed thereby preventing any distal flow of blood through the flow channels 16a. FIG. 9 shows a cross section of the device embodiment with the nosecone 24 including a “rapid exchange” configuration where the guidewire 57 enters the distal tip port 54 of the nosecone 24 and exits the proximal end of the nosecone 24 at proximal port 53 distal of the inflatable balloon 15 and bypasses the remainder of the device until it reaches an alignment hole 25 disposed within a guidewire clip 22. By passing the guidewire 57 through the alignment hole 25, it eliminates the potential that the deployment rods 39 and anchors 28 are deployed on opposite sides of the guidewire 57 (thereby entrapping it) which might result in the inability to remove the inner catheter assembly 10 from the patient without first removing the guidewire 57. In some cases, the alignment hole 25 may include a slotted lumen configured to releasably secure the guidewire 57 therein under normal lateral loads but allow ingress and egress of the guidewire 57 through a lateral slot thereof when lateral loads larger than nominal lateral loads are applied by a user. For such embodiments a resilient snap-type fit may be achieved.

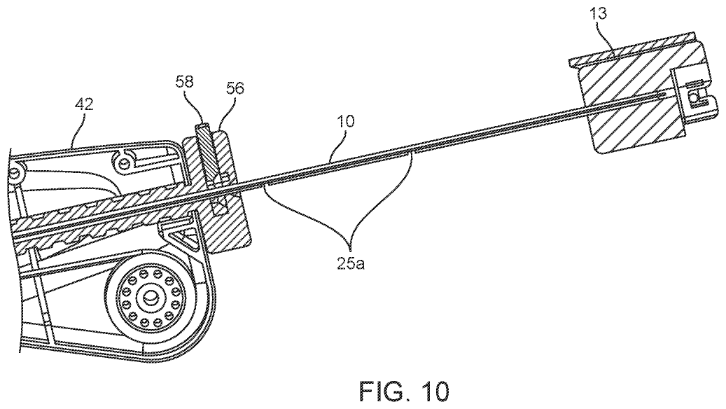

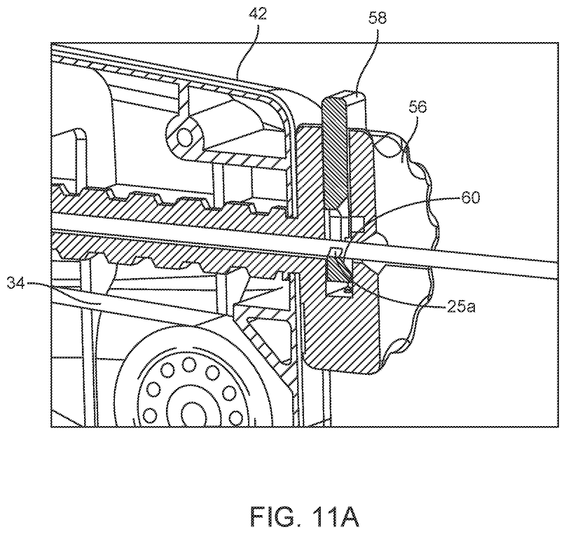

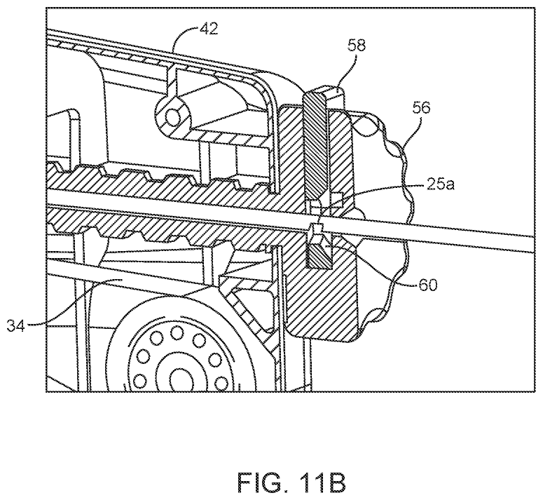

FIG. 10 shows the notches or detents 25a in the elongate shaft 46 of the inner catheter assembly embodiment 10. These notches 25a, which may be disposed on only one side of the elongate shaft 46 of the inner catheter assembly 10, may be configured to provide a definitive stop when axially sliding the chassis 42 down the elongate shaft 46 of the inner catheter assembly 10 and when withdrawing the inner catheter assembly embodiment 10 from the artery when the knob 56 is in the vertical position and the spring loaded detent tab 58 is facing upwards. FIG. 11A shows the action of the detent interlock 60 to define the location of the inner catheter assembly 10 relative to the chassis 42. The springs forces the interlock 60 up into the inner catheter assembly 10 cutouts. FIG. 11B shows pressing the detent interlock 58 (against the springs) disengages the interlock 60 to allow the inner catheter assembly 10 to move to its next position. It should be noted that because the notches 25a are disposed on only one side of the elongate shaft 46 and the detent tab 58 is also on one side of the knob, the notches 25a and detent tab 58 must be rotationally aligned in order for the detent interlock 60 to function. Therefore, if the knob 56 is rotated away from the rotational alignment as shown in FIGS. 11A and 11B by a suitable amount, such as by 180 degrees, the elongate shaft 46 and detents 25a thereon will be able to freely translate within the knob 56 and detent interlock 60.

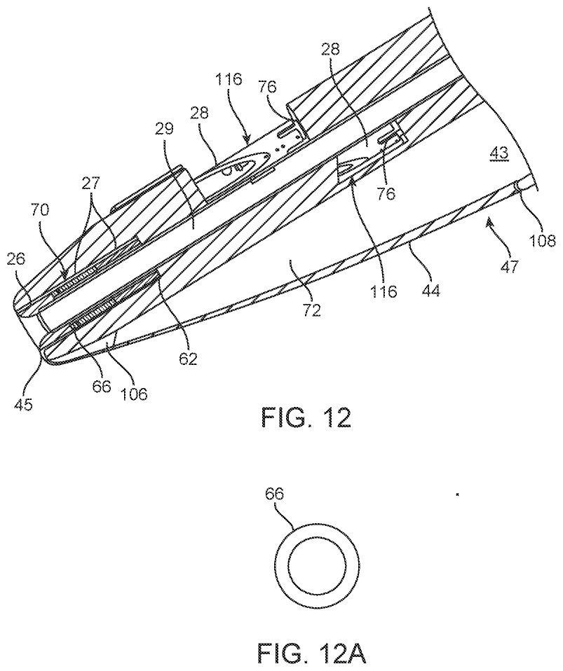

FIG. 12 shows a nosetip embodiment of the elongate housing embodiment 44. Contained within the nosetip are the filament fairlead sleeve 26, filament lock 27, two pieces comprise the filament lock, anchors 28 (four places), and filament tube 29. The four anchors 28 are attached to four filaments 40. These filaments 40 may be fed through the filament tube 29 and then attached to a constant force spring 34 with a simple quick disconnect such as a tension transfer clip 34a in some instances. The filament lock 27 may be engaged onto the filaments 40 by retracting the filament tube 29 from the filament lock 27 and allowing the filament lock tangs to spring inward to grab the filaments 40. Since there is a backstop surface 62 for the filament lock elements 27, they are configured to translate axially along the filament tube 29 when they are pulled back in a proximal direction against the backstop.

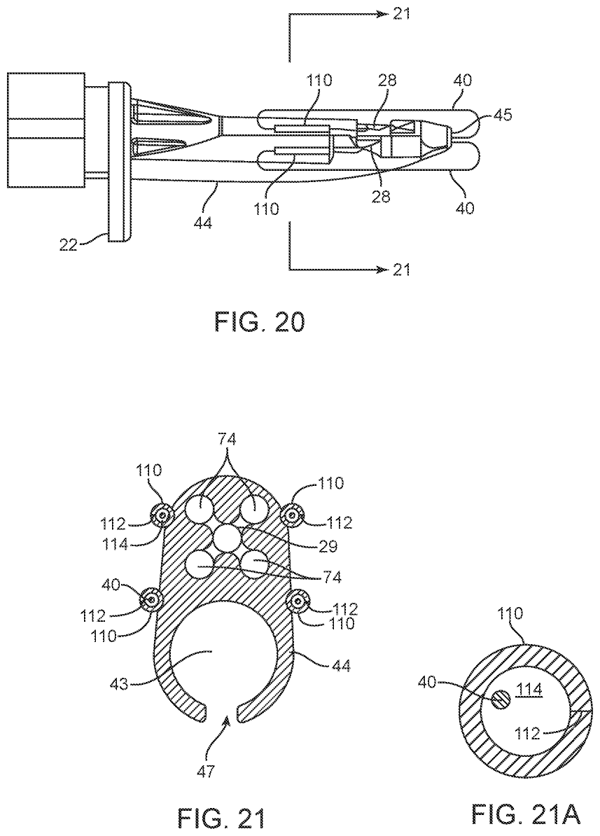

Further regarding embodiments of the elongate housing, and with reference to FIGS. 1-6, 12, 20 and 21, some vascular closure assembly embodiments 8 may include an inner catheter assembly 10 including the elongate shaft 46 having a proximal end, a distal end 52, a distal section, an axial length and a guidewire lumen 55. For some embodiments, the guidewire lumen extends proximally from a distal port 54 at the distal end 52 of the elongate shaft 46 to a proximal port 53 disposed at the distal section as shown in FIG. 9. The vascular closure assembly 8 may also include the actuator assembly 9 having a chassis portion 42 and a plurality of anchor deployers 68, each anchor deployer 68 including a deployment rod 39, an anchor 28 which may be removably secured to a distal end of the deployment rod 39, and a filament 40 secured to each anchor 28. An elongate housing 44 of the actuator assembly 9 may have a proximal end secured to a distal end of the chassis portion 42 and include a distal end 45 and an inner lumen 43 extending along the elongate housing 44 to the distal end 45 of the elongate housing 44. The inner lumen 43 may include an inner surface contour which is configured to be slidably disposed over an outer surface of the elongate shaft 46 of the inner catheter assembly 10.

The elongate housing embodiment 44 may also include a plurality of anchor deployer lumens 74 configured to be slidably disposed about a respective anchor deployer 68, each anchor deployer lumen 74 extending axially along the elongate housing 44, or along any other suitable path along the elongate housing 44, and terminating distally at the distal port 76 disposed in the distal section 72 of the elongate housing 44. Some embodiments of the elongate housing 44 may further include a guidewire relief slot 47 which is disposed in the inner lumen 43 through a wall portion thereof, which extends proximally from the distal end 106 of the inner lumen 43 to a proximal end 108 of the guidewire relief slot 47, and which is configured to accommodate a guidewire extending outwardly from the proximal port 53 of the guidewire lumen 55 of the elongate shaft 46. In addition, in some cases, a guidewire retention clip 22 which extends outwardly from the elongate housing 44 may be disposed proximally of the proximal end 108 of the guidewire relief slot 47.

Some vascular closure assembly embodiments 8 may include the actuator assembly 9 having a chassis portion 42 and a plurality of anchor deployers 68, each anchor deployer 68 including the deployment rod 39, the anchor 28 which may be removably secured to a distal end of the deployment rod 39, and the filament 40 secured to each anchor 28. The actuator assembly 9 of such embodiments may also include the elongate housing 44 having a proximal end secured to a distal end of the chassis portion 42, a distal end 45, and the inner lumen 43 extending along the elongate housing 44 to the distal end 45 of the elongate housing 44. The elongate housing may also include a plurality of anchor deployer lumens 74 configured to be slidably disposed about the respective anchor deployer 68, each anchor deployer lumen 74 extending axially along the elongate housing 44, or along any other suitable path along the elongate housing 44, and terminating distally at the distal port 76 disposed in the distal section of the elongate housing 44. The elongate housing may also include a plurality of filament retainers 110 disposed proximally of the distal ports 76 of the anchor deployer lumens 74, each filament retainer 110 being configured to releasably secure a section of a respective filament 40. In some instances, each of the filament retainers 110 may include a split tube configuration which may include a tubular structure with a split 112 in the wall structure thereof that extends completely through the wall of the tubular structure and extends along an entire axial length of the tubular structure.

For such filament retainer embodiments 110, if made from a pliable and resilient material with an inner lumen 114 disposed about a respective filament 40 and a split 112 extending an axial length of the split tube 110, a filament 40 disposed within the inner lumen 114 may be releasably secured therein under normal use and tissue interaction during deployment and positioning of the vascular closure assembly 8, but release the filament 40 when under loads associated with deployment of the anchor deployers 68 and subsequent tensioning of the filaments 40. For some embodiments, the split tubes of some filament retainer embodiments 110 may include polymer having a durometer range of about 20 Shore D to about 80 Shore D. or the like. In some cases, the elongate housing 44 may further includes a plurality of anchor pockets 116. The anchor pocket 116 may be disposed adjacent a respective distal port 76 of the anchor deployer lumens 74 and be configured to accept a respective anchor 28 so as to allow a sharpened distal tip of the anchor 28 to be disposed below a nominal outer surface profile of the distal section of the elongate housing 44 while the anchor deployers 68 are in an undeployed state.

Some vascular closure assembly embodiments 8 may include the chassis 42, the elongate housing 44 which has a proximal end thereof secured to a distal end of the chassis 42 and a plurality of anchor deployers 68 which are configured to extend from a distal section of the elongate housing 44, each anchor deployer 68 including an anchor 28 and a filament 40 secured to the anchor 28. The vascular closure assembly 8 may further include a filament lock assembly 70 including the filament tube 29, and one or more filament locks 27. The filament locks 27 may include an inner lumen, the inner lumen being disposed over an outer surface of a distal section of the filament tube 29. The filament lock assembly 70 may further include the fairlead sleeve 26 which has an inner lumen disposed over the filament tube 29 and disposed axially adjacent and distal of the filament lock 27. Such an embodiment may further include a polymer bushing 66 disposed between the fairlead sleeve 26 and the adjacent filament lock 27. In some cases, the polymer bushing 66 may include a polymer such as nylon, polyimide or the like. Such a polymer bushing 66 may be configured to prevent ohmic contact and possible electrolysis between the filament lock 27 and the axially adjacent fairlead sleeve 26 which may be made from a different metallic material from that of the filament lock 27 in some cases.

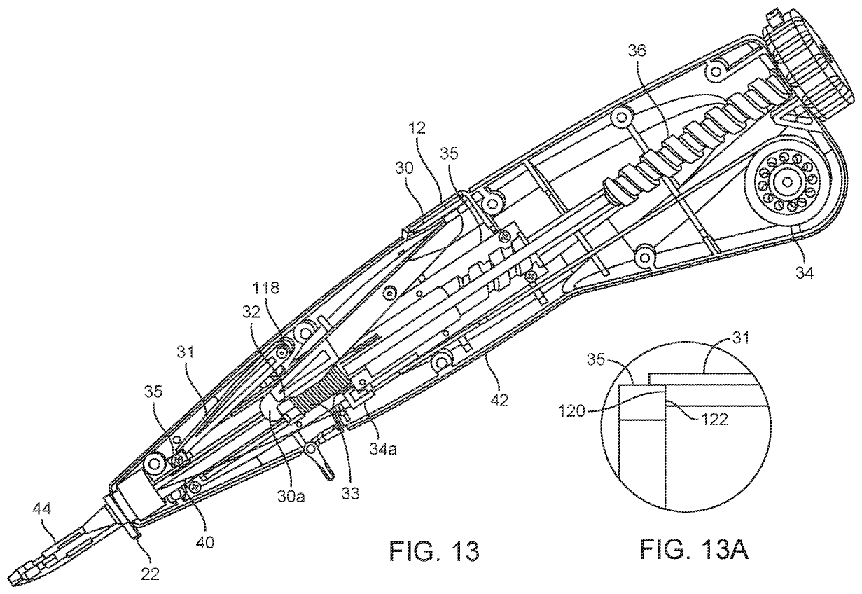

FIG. 13 shows a cross sectional view of the chassis embodiment 42 in a cocked loaded position where it is ready for deployment. A platen catch 30a of the platen latch 30 captivates an deployment rod platen 32. The deployment rod platen 32 can be driven forward by a compression spring 33 when the platen latch trigger 30 is pressed or otherwise actuated. The tensioner that may include the constant force spring 34 is operatively coupled to a plunger 35 by means of the tension transfer clip 34a. The plunger 35 is kept in the loaded position by a trigger latch lever 31. When the deployment rod platen 32 is driven forward by the compression spring 33, the deployment rods 39 with anchors 28 attached are driven through the fascia tissue layer 64 as shown in FIG. 17. For some embodiments, when the deployment rod platen 32 reaches the end of its travel it pushes up on the latch lever 31 and disengages it from the plunger 35, allowing the plunger 35 to be pulled back by the constant force spring 34 until the plunger 35 engages the thumb screw 36.

Some vascular closure assembly embodiments 8 may include the actuator assembly 9 which may include the chassis 42 with a distal end and a proximal end. The plunger 35 may be proximally translatable with respect to the chassis 42 over a retraction length starting from a distal position of the plunger 35 as shown in FIG. 13. The tensioner 34 has a first end secured to the chassis 42, a second end releasably secured to the plunger 35 with the tension transfer clip 34a and is configured to continuously apply proximally oriented tension to the plunger 35 with respect to the chassis 42 over the retraction length of the deployment rods 39 and filaments 40. A trigger latch 31 may be configured to releasably secure the plunger 35 in the distal position in opposition to the proximal force applied to the plunger 35 by the tensioner 34. The platen 32 may be distally translatable with respect to the plunger 35 from a proximal cocked position over a deployment length to a distal position that actuates the trigger latch 31 and releases the plunger 35 allowing proximal translation of the plunger 35 over the retraction length.

A compression spring 33 which has a first end operatively coupled to the plunger 35 and a second end operatively coupled to the platen 32 may be configured to apply a distally oriented force to the platen 32 from the proximal cocked position of the platen 32 over the deployment length to the distal position of the platen 32. In some instances, the platen 32 translates in a constrained linear movement relative to the plunger 35 upon actuation or release of the compression spring 33. A platen latch 30 may be operatively coupled to the chassis 42 with a configuration allowing actuation of the platen latch 30 but preventing distal translation of the platen latch 30 with respect to the chassis 42. The platen latch 30 may include a platen catch 30a that is operatively coupled to the platen 32 and releasably secures the platen 32 in a proximal cocked position. The actuation or deployment button 12 may be operatively coupled to the platen latch 30 and configured to actuate the platen latch 30 to disengage the platen catch 30a from the platen 32.

The actuator assembly 9 may further include the elongate housing 44 which has a proximal end thereof secured to a distal end of the chassis 42 and a plurality of anchor deployers 68. Each anchor deployer 68 may be slidably disposed within a respective anchor deployer lumen 74 of the elongate housing 44. In some cases, each anchor deployer 68 may include the deployment rod 39 having an elongate resilient configuration which is operatively coupled to the platen 32 such that distal translation of the platen 32 results in distal translation of the deployment rod 39. An anchor 28 may be removably secured to the distal end of the deployment rod 39 in some instances. In some cases, the plunger 35 may include a tubular configuration that is constrained to translate proximally in a linear axial direction relative to the chassis 42 from the distal position of the plunger 35. The platen 32 may be disposed and axially translatable within an inner lumen of the tubular plunger 35 in some instances. For some embodiments, a proximal section of the plunger 35 may include a threaded barrel section.

For some embodiments, the trigger latch 31 may include a pivoting configuration having a proximal end 118 which is pivotally coupled to the chassis 42 and a distal end which includes a distal facing engagement surface 120 that engages a proximal facing latch surface 122 of the plunger 35. For some embodiments, the tensioner 34 may include a constant tension spring such as a wound ribbon shaped clock spring or the like. For some embodiments, the compression spring 33 may include a helically wound cylindrically shaped or conically shaped spring.

For such actuator assembly embodiments 9, a method of actuating the actuator assembly 9 may include actuating the platen latch 30 of the actuator assembly 9 with the actuation button 12 which is operatively coupled to a chassis 42 thereby releasing the compression spring 33, which is operatively coupled between a plunger 35 and a platen 32, from a compressed state of the compression spring 33. Thereafter, axially translating the platen 32 and associated deployment rods 39 which are operatively secured thereto in a distal direction relative to the plunger 35 and chassis 42 under a distal force generated by the released compression spring 33. The trigger latch 31 may then be actuated with the platen 32 as it translates distally, thereby subsequently releasing the plunger 35 from the fixed distal position. Thereafter, the method may include axially translating the plunger, platen and deployment rods secured to the platen in a proximal direction under a proximal force generated by the tensioner 34 which is secured to the chassis 42 and releasably secured to the plunger 35 with the tension transfer clip 34a.

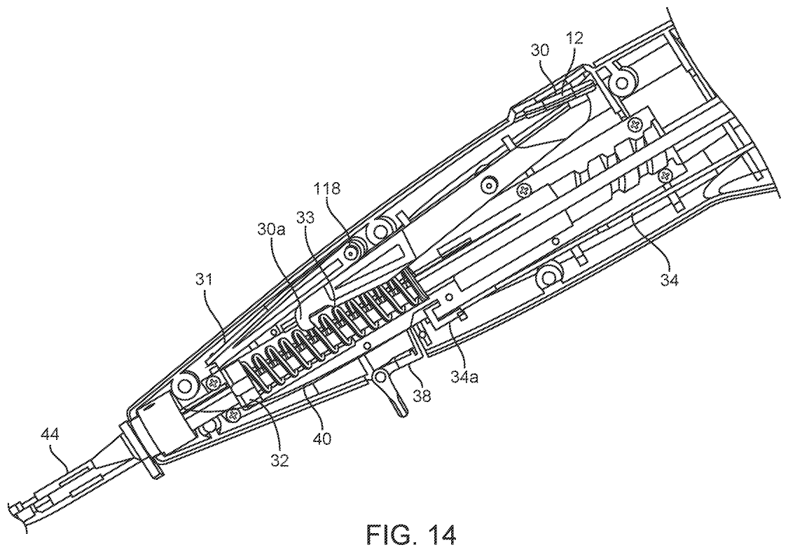

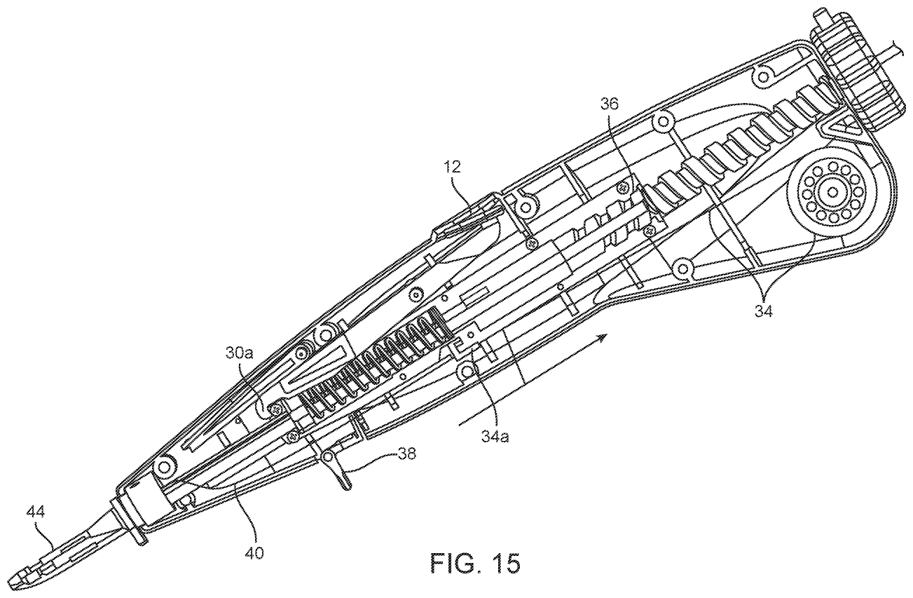

FIG. 14 shows the initial part of an anchor deployment embodiment where the platen latch 30 has been depressed, which results in the compression spring 33 forcing the deployment rod platen 32 to move distally, which, releases the latch lever 31. When the deployment rod platen 32 moves distally, it may be configured to drive the four anchors 28 distally to a position below the tissue layer 64 as shown in FIG. 17, as the anchors 28 are attached to the ends of the deployment rods 39. FIG. 15 shows a cross sectional view of the second portion of the deployment sequence. After the latch lever 31 has been lifted by the deployment rod platen 32, the plunger 35 slides proximally until the plunger 35 engages the thumb screw 36. During this sequence, as the deployment rod platen 32 slides proximally, it may be configured to withdraw the deployment rods 39 from the tissue layer 64 leaving the anchors 28 (with filaments connected) below the tissue layer 64.

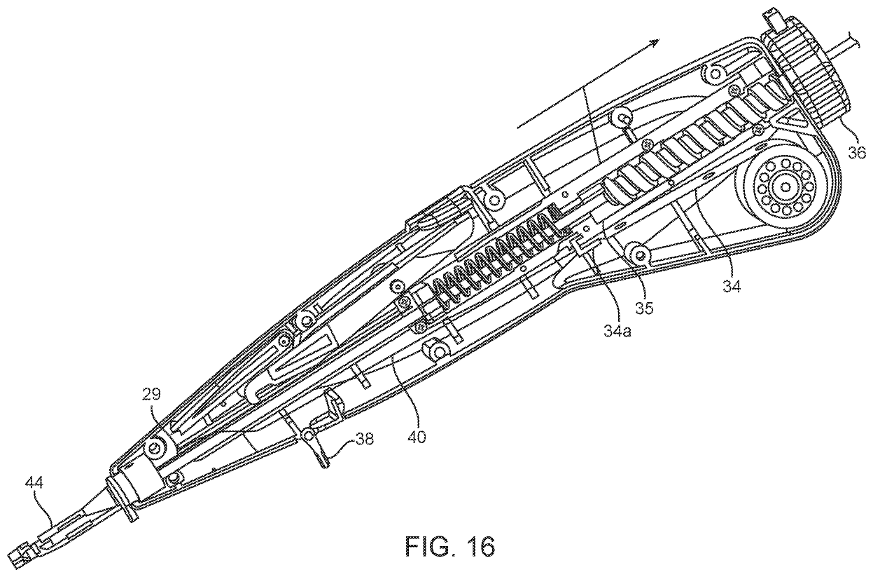

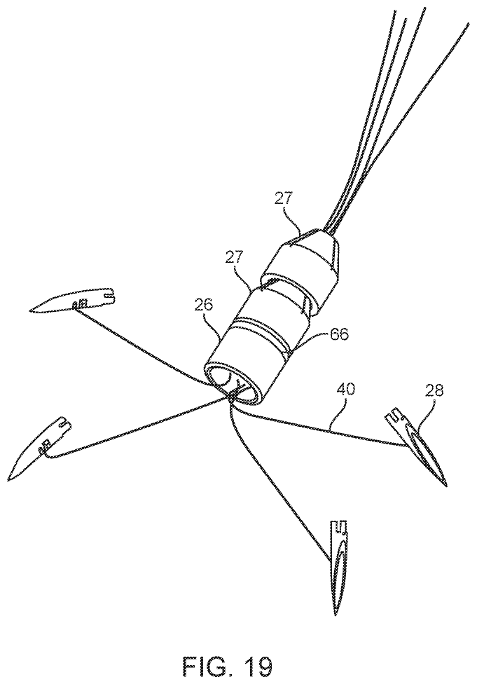

FIG. 16 shows the next steps in the deployment sequence embodiment. Once the plunger 35 engages the thumb screw 36, the thumb screw 36 may be turned which continues the retraction of the plunger 35 in a controlled manner. The filaments 40 are attached to the constant force spring 34 through the tension transfer clip 34a. Once the tension in the filaments 40 reaches equilibrium with the constant force spring 34, the tension transfer clip 34a and associated tensioner 34 disengages from the plunger 35. As the plunger 35 is retracted by further rotation of the thumb screw 36, at a specified distance, the end of the filament tube 29 is engaged resulting in the filament tube 29 retracting (moving proximally) from the filament lock 27, allowing the filament lock 27 to engage the filaments 40 as shown in FIG. 19. Finally, the filaments 40 are cut, disconnecting them from from the constant force spring 34 by actuation of the filament cutter 38 which is shown in FIG. 14-16. The filament cutter 38 is configured to be actuated as a separate step by pulling rearward on the lever of the filament cutter 38 that extends downward from the chassis 42. Pulling rearward on the lever of the filament cutter 38 pivots a filament cutter blade (not shown) upward and into the adjacent tensioned filaments 40 (not shown) thereby cutting them and allowing withdrawal of the vascular closure assembly 8.

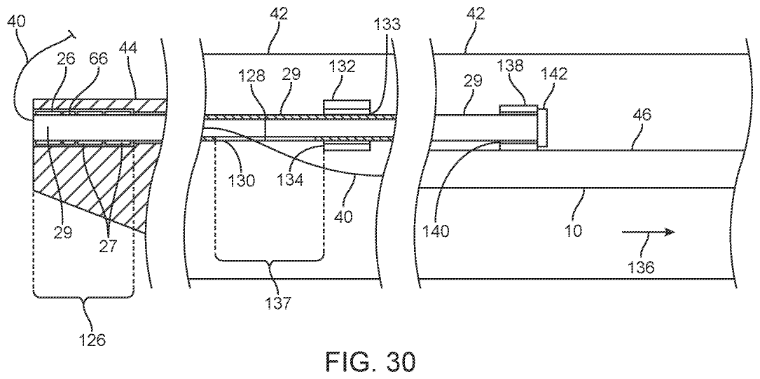

For some vascular closure assembly embodiments 8, deployment of the filament locks 27 and subsequent cutting of the filaments 40 may be carried out in sequence with one action by the operator by actuation/retraction of the filament tube 29. FIGS. 30-32 show a schematic representation of a nosetip of the elongate housing 44 and chassis 42 in partial section and with multiple break aways that illustrate an embodiment for carrying out such a process. FIG. 30 shows the filament tube 29 disposed in a distal-most position with the filaments locks 27, filament sleeve 26 and busing 66 disposed over a distal end section of the filament tube 29 within the elongate housing 44. The axial length of engagement 126 of the distal end section of the filament tube 29 with the filament locks 27, filament sleeve 26 and busing 66 is represented by bracket 126. The axial length of engagement 126 represents the amount of proximal retraction of the filament tube 29 necessary to fully deploy the filaments locks 27, filament sleeve 26 and bushing 66 from the filament tube 29.

Also shown in FIG. 30 is a mid-section of the filament tube 29 which includes an elongate passage 128 through a wall portion of the filament tube 29, the distal end of the elongate passage 128 including a first cutting edge 130. The mid-section of the filament tube 29 passes through a lumen 133 of a cutting block 132 which has a second cutting edge 134 disposed at a distal end thereof. The cutting block 132, lumen 133 and associated first cutting edge 130 may take any suitable form such as a tubular member having a sharpened distal end. In some cases, an outside surface of the filament tube 29, an inside surface of the lumen 133 and the first and second cutting edges 130, 134 may be configured to create a shear cutting function when the filament tube 29 is proximally retracted, as indicated by arrow 136, such that the first cutting edge 130 and second cutting edge 134 come together and eventually translate past each other. The axial separation between the first cutting edge 130 and second cutting edge 134 shown in FIG. 30 with the filament tube 29 in the distal most position may be referred to as the cutting stroke length as indicated by the bracket 137. In order to achieve a proper sequential deployment of the filament anchors 27 and subsequent cutting of the filaments 40, it may be important in some cases for the cutting stroke length 137 to be greater than the axial length of engagement 126.

FIG. 30 also shows a schematic representation of a proximal portion of the chassis 42 that illustrates a coupling embodiment between the filament tube 29 and elongate shaft 46 of the inner catheter assembly 10. The coupling embodiment therebetween includes a tension block 138 secured to the elongate shaft 46 and having an inner lumen 140 that is configured to be slidably disposed over an outer surface of the nominal section of the filament tube 29. A tab 142 is secured to the filament tube 29 proximally of the tension block 138 and includes a transverse dimension that is too large to fit through the inner lumen 140 such that as the elongate shaft 46 and associated tension block 138 are proximally retracted by the operator, the filament tube 29 will slide within the lumen 140 of the tension block 138 until contact is made between the tab 142 of the filament tube 29 and the tension block 138. Thereafter, further proximal retraction of the elongate shaft 46 and tension block 138 as indicated by arrow 136 will impart a proximal retraction force to the tab 142 and filament tube 29.

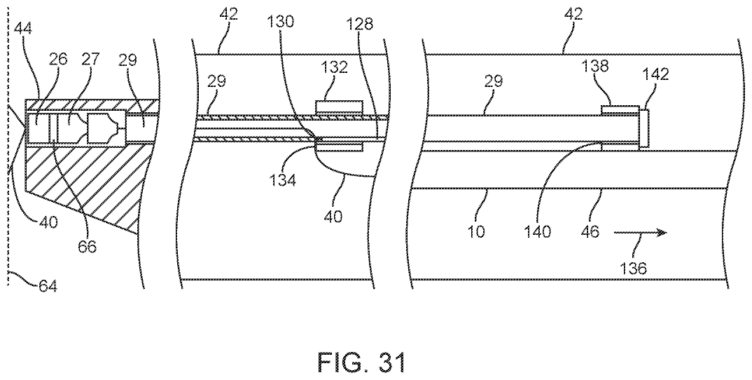

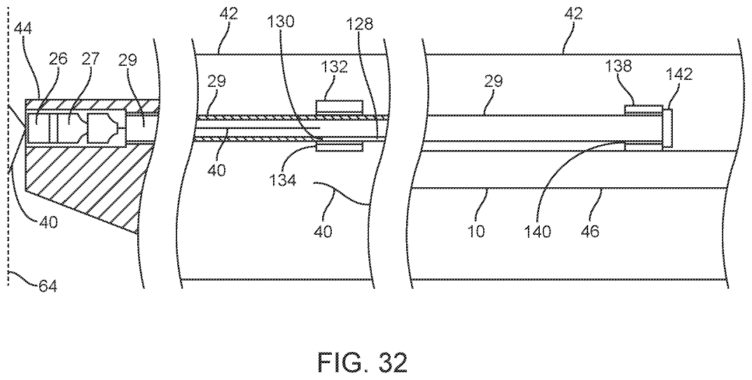

FIG. 31 illustrates the effect of proximal retraction of the filament tube embodiment 29 shown in FIG. 30 by means of proximal retraction of the elongate shaft 46. In FIG. 31, the filament tube 29 has been proximally retracted such that the distal section and distal end of the filament tube 29 has been fully retracted from the filament locks 27, filament sleeve 26 and bushing 66 thus fully deploying these elements onto the sutures 40 as shown. The proximal retraction of the filament tube 29 has also brought the first cutting edge 130 into close proximity to the second cutting edge 134 with the filaments 40 passing from the inner lumen of the filament tube 29 therebetween to a position within the chassis 42 but outside the lumen of the filament tube 29. As such, in FIG. 31, the filament locks 27, filament sleeve 26 and filament bushing 66 have been fully deployed and the first cutting edge 130 and second cutting edge 134 are poised to sever the filaments 40 upon further proximal retraction of the filament tube 29. FIG. 32 illustrates the mid-section of the filament tube 29 and cutting block 132 after further proximal retraction of the filament tube 29 subsequent to the relative position shown in FIG. 31. In FIG. 32, the filaments 40 have been cut and the first cutting edge 130 is now disposed proximally of the second cutting edge 134.

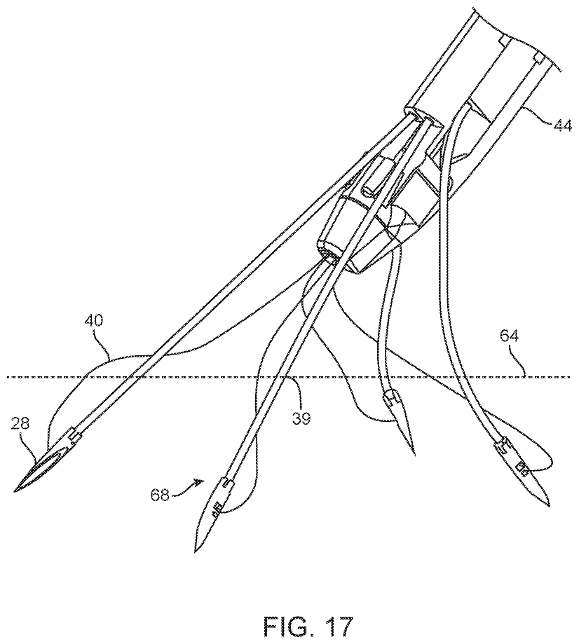

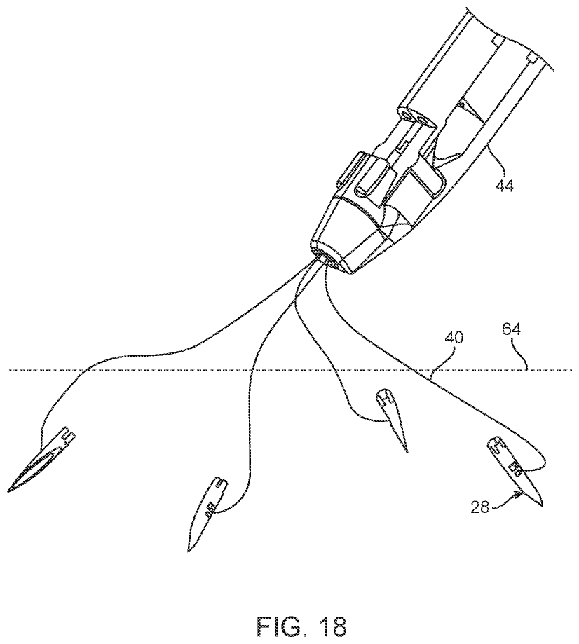

FIG. 17 shows the distal end of the nosetip of the elongate housing embodiment 44 with the deployment rods 39 distally extended and the anchors 28 attached to the end of these deployment rods 39. FIG. 17 also shows the filaments 40 that are attached to the anchors 28 and the way the filaments 40 are fed up through the centrally located filament tube 29. FIG. 18 shows the anchors 28 deployed. The tissue layer 64 and passage therethrough are not shown, but the anchors 28 would be engaged below the tissue layer 64 and the anchors 28 may be circumscribed around the passage. FIG. 19 shows the components of the device that remain inside the patient—the implant. The filament lock 27 is in a deployed state with the locking tabs sprung inward onto the filaments 40, thus engaged into the four filaments 28 so as to clamp them together preventing relative movement between the clamped portions of the four filaments 40 and locking tabs and preventing them from loosening. The four anchors 28 that are attached to the filaments 40 would be pulled taught, thus creating a bunching of tissue layer 64 (not shown) disposed over the passage that results in plugging of the access hole in the artery.

As discussed above, some vascular closure assembly embodiments 8 may include anchor deployers 68 that include deployment rods 39 having a pre-shaped or curved configuration which may include a smooth continuous curvature in some cases. Some such vascular closure assembly embodiments 8 may include the actuator assembly 9 having a chassis portion 42 and the elongate housing 44 with a proximal end thereof secured to a distal end of the chassis portion 42, a distal end extending away from the chassis portion 42, a distal section 72 that may include the nosetip, and a plurality of anchor deployer lumens 74. In some cases, each anchor deployer lumen 74 may extend axially along the elongate housing 44 and terminate distally at a distal port 76 disposed in the distal section 72 of the elongate housing 44.

A plurality of the anchor deployers 68 may each be slidably disposed within a respective anchor deployer lumen 74 of the elongate housing 44. Each anchor deployer 68 may include the deployment rod 39 which includes an elongate resilient configuration, and a pre-shaped distal section 78 that assumes a curved profile while in a relaxed state and a straightened profile while in a constrained state within the respective anchor deployer lumen 74 and that is configured to extend from the respective distal port 76 along a curved path as an extended portion thereof relaxes and assumes the curved profile. Each of the anchor deployers 68 also includes an anchor 28 which is removably secured to the distal end of the deployment rod 39 with some anchor embodiments being configured to resist proximal retraction within tissue. A filament 40 may be secured to each anchor 28. For some such embodiments, the pre-shaped distal section 78 may have a pre-shaped profile that lies in a plane without compound curvature.

For some embodiments the pre-shaped distal section 78 of each deployment rod 39 is configured to extend distally from the respective distal port 76 until a distal end of the deployment rod 39 is disposed at a tissue penetrating position at a tissue penetrating angle with a distal end of the elongate housing 44 disposed adjacent the tissue layer 64 with a longitudinal axis of the elongate housing 44 being disposed at a tilted deployment angle with respect to the tissue layer 64. For some embodiments, the elongate housing 44 and pre-shaped distal section 78 of each of the deployment rods 39 are configured to extend the deployment rods 39 and engage the tissue layer 64 at a tissue penetrating angle with the elongate housing 44 disposed at a deployment angle of about 40 degrees to about 50 degrees with respect to the patient.

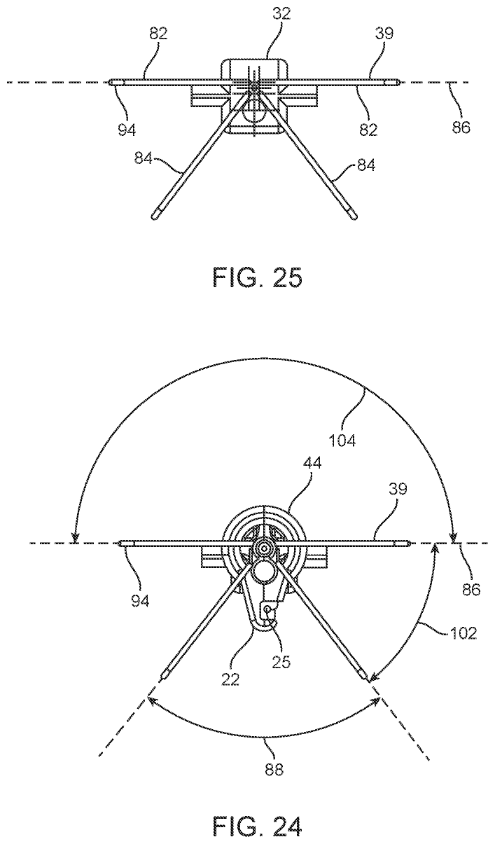

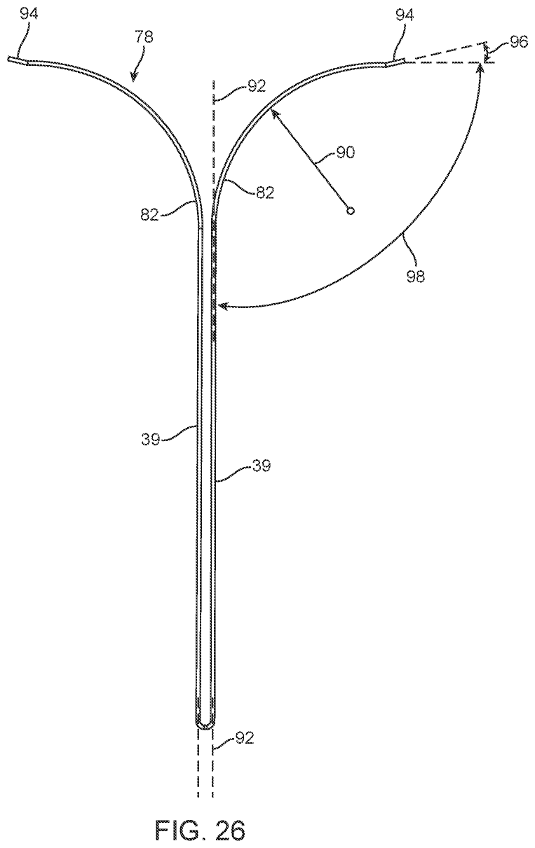

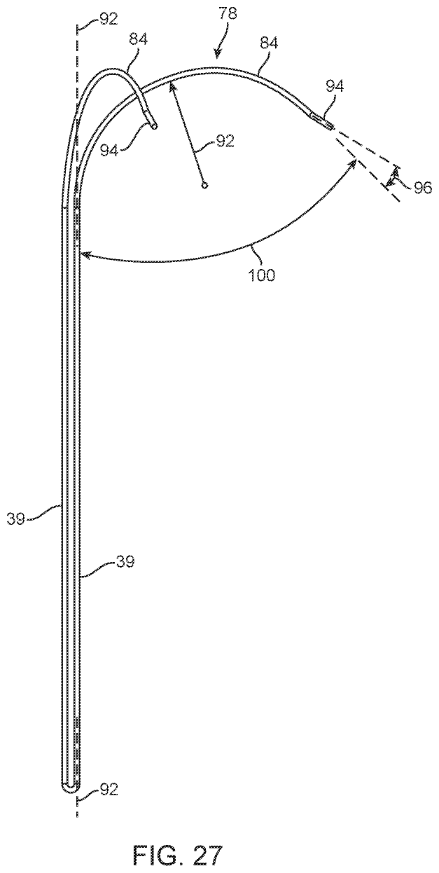

In some cases, the deployment rods 39 of the plurality of anchor deployers 68 may include at least two cranial deployment rods 82, the distal tips of which extend laterally away from the distal section 72 of the elongate housing 44 and each other as shown in FIG. 26. The plurality of anchor deployers 68 may also include at least two caudal deployment rods 84 that extend away from the distal section of the elongate housing 44 and below the cranial deployment rods 82. In some instances, the pre-shaped distal sections 78 of the at least two cranial deployment rods 82 lie in the same plane 86 when in an extended deployed state as shown in FIGS. 24 and 25 forming a relative angle 104 therebetween of about 180 degrees but may be about 160 degrees to about 200 degrees. For some embodiments, the pre-shaped distal sections 78 of the at least two caudal deployment rods 84 lie in respective planes disposed at an angle 88 of about 70 degrees to about 125 degrees, more specifically, about 70 degrees to about 110 degrees, to each other as shown in FIG. 24. For some embodiments, a relative angle 102 disposed between a plane of the pre-shaped distal section 78 of the cranial deployment rod 82 and a plane of the pre-shaped distal section 78 of an adjacent caudal deployment rod 84 may be about 30 degrees to about 75 degrees, more specifically, about 30 degrees to about 55 degrees. In some cases, a radius of curvature 90 of the pre-shaped distal sections 78 of the cranial deployment rods 82 as shown in FIG. 26 may be about 22 mm to about 30 mm. For some such embodiments, a radius of curvature 92 of the pre-shaped distal sections 78 of the caudal deployment rods 84 may be about 12 mm to about 18 mm as shown in FIG. 27.

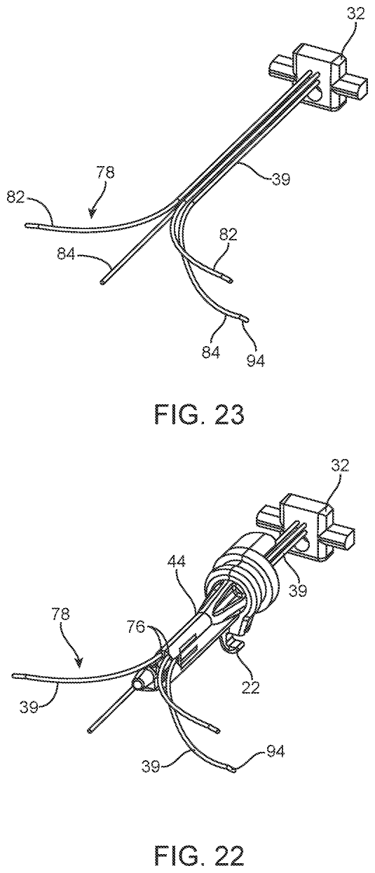

For some embodiments, the deployment rods 39 may be configured to translate axially with respect to the elongate housing 44 but be fixed with respect to rotation about their respective longitudinal axes 92. FIG. 22 shows the four deployment rods 39 with respective proximal ends thereof secured to the platen 32 such that rotation of the deployment rods 39 about their longitudinal axis 92 with respect to the platen 32, chassis 42 and elongate housing 44 is prevented. The deployment rod embodiments 39 shown in FIGS. 22-27 include an anchor engagement section 94 that extends proximally from the distal end of the deployment rod 39 and is angled in a direction opposite that of the curved profile of the pre-shaped distal section 78. In some cases, the anchor engagement section 94 of each deployment rod 39 extends proximally from the distal end of the deployment rod 39 by a distance of up to about 0.5 to about 1.5 times an axial length of the anchor 28. For some embodiments, the anchor engagement section 94 of each deployment rod 39 is angled opposite to the curved profile of the pre-shaped distal section 78 by an angle 96 by about 16 degrees to about 22 degrees.

For some embodiments, the pre-shaped distal sections 78 of the cranial deployment rods 82 are configured to include a nominal distal tip angle 98 (without the anchor engagement section 94) of about 80 degrees to about 90 degrees with respect to the longitudinal axis 92 of the deployment rod 39 which is disposed proximal of the pre-shaped distal section 78 while the pre-shaped distal section 78 is disposed in a relaxed unconstrained state as shown in FIG. 26. In some instances, the pre-curved distal sections 78 of the caudal deployment rods 84 are configured to include a nominal distal tip angle 100 (without the anchor engagement section 94) of about 110 degrees to about 130 degrees with respect to the longitudinal axis 92 of the deployment rod 39 which is disposed proximal of the pre-shaped distal section 78 while the pre-shaped distal section 78 is disposed in a relaxed unconstrained state as shown in FIG. 27. In some cases, the pre-shaped distal sections 78 of the cranial deployment rods 82 may be configured to include a lateral displacement of the distal tip of the deployment rod 39 from the longitudinal axis 92 of the deployment rod 39 normal to the distal tip of about 20 mm to about 30 mm with the pre-shaped distal section 78 of the deployment rod 39 in a relaxed unconstrained state. In addition, the pre-shaped distal sections 78 of the caudal deployment rods 84 may be configured to include a lateral displacement of the distal tip of the deployment rod 39 from the longitudinal axis 92 of the deployment rod 39 normal to the distal tip of about 20 mm to about 30 mm with the pre-shaped distal section 78 of the deployment rod 39 in a relaxed unconstrained state.

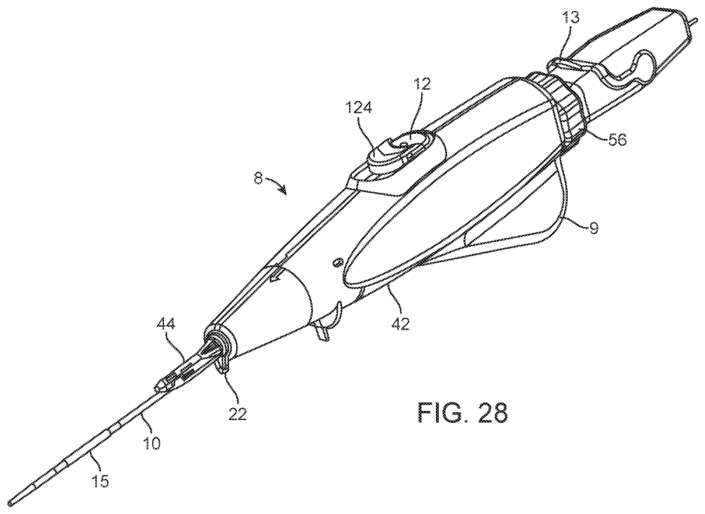

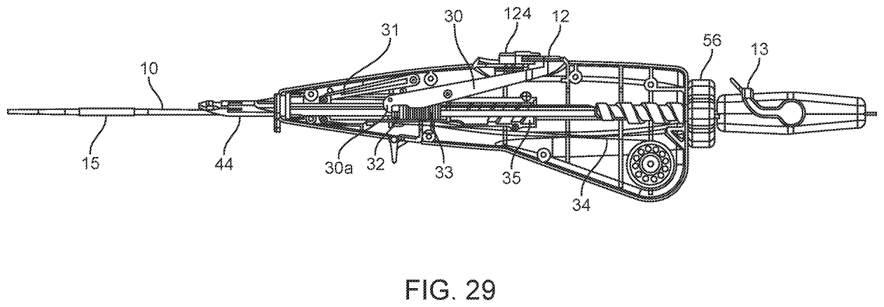

FIGS. 28 and 29 show an embodiment of a vascular closure assembly 8 that may have the same or similar features, dimensions or materials as those of the vascular closure assembly embodiments 8 discussed above. The actuator assembly 9 of the embodiment shown may include an actuator button 12 and associated trigger latch 31 that releasably restrains the platen 32 as discussed above. The actuator assembly 9 also includes a deployment button cover 124 that is configured to slide relative to the chassis 42 and mechanically capture the actuator button 12 to prevent inadvertent actuation of the assembly. In order to arm the actuator assembly 9, the deployment button cover 124 may be slid distally in order to free the actuator button 12 to allow movement and actuation thereof.

Embodiments illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation and use of such terms and expressions do not exclude any equivalents of the features shown and described or portions thereof, and various modifications are possible. The term “a” or “an” can refer to one of or a plurality of the elements it modifies (e.g., “a reagent” can mean one or more reagents) unless it is contextually clear either one of the elements or more than one of the elements is described. Thus, it should be understood that although embodiments have been specifically disclosed by representative embodiments and optional features, modification and variation of the concepts herein disclosed may be resorted to by those skilled in the art, and such modifications and variations are considered within the scope of this disclosure.

With regard to the above detailed description, like reference numerals used therein refer to like elements that may have the same or similar dimensions, materials and configurations. While particular forms of embodiments have been illustrated and described, it will be apparent that various modifications can be made without departing from the spirit and scope of the embodiments of the invention. Accordingly, it is not intended that the invention be limited by the forgoing detailed description.

What is claimed is:

1. A vascular closure assembly, comprising:
an actuator assembly including:
a chassis portion;
an elongate housing with a proximal end thereof secured to a distal end of the chassis portion, a distal end extending away from the chassis portion, a distal section, and a plurality of anchor deployer lumens, each anchor deployer lumen extending along the elongate housing and terminating distally at a distal port disposed in the distal section of the elongate housing; and
a plurality of anchor deployers, each anchor deployer being slidably disposed within a respective anchor deployer lumen of the elongate housing, each anchor deployer comprising:
a deployment rod which includes an elongate resilient configuration, and a pre-shaped distal section that assumes a curved profile while in a relaxed state and a straightened profile while in a constrained state within the respective anchor deployer lumen and that is configured to extend from the respective distal port along a curved path as an extended portion thereof relaxes and assumes the curved profile,
an anchor which is removably secured to the distal end of the deployment rod, and which is configured to resist proximal retraction within tissue; and
a filament secured to each anchor.

2. The vascular closure assembly of claim 1 wherein the pre-shaped distal section of each deployment rod is configured to extend distally from the respective distal port until a distal end of the deployment rod is disposed at a tissue penetrating position at a tissue penetrating angle with a distal end of the elongate housing disposed adjacent a tissue layer with a longitudinal axis of the elongate housing being disposed at a tilted deployment angle with respect to the tissue layer.

3. The vascular closure assembly of claim 2 wherein the elongate housing and pre-shaped distal section of each of the deployment rods are configured to extend the deployment rods and engage the tissue layer at a tissue penetrating angle with the elongate housing disposed at a deployment angle of about 40 degrees to about 50 degrees with respect to the patient.

4. The vascular closure assembly of claim 1 wherein the pre-shaped distal section of each deployment rod lies in a plane

5. The vascular closure assembly of claim 1 wherein the deployment rods of the plurality of anchor deployers comprise at least two cranial deployment rods, the distal tips of which extend laterally away from the distal section of the elongate housing and each other, and at least two caudal deployment rods that extend away from the distal section of the elongate housing and below the cranial deployment rods.

6. The vascular closure assembly of claim 5 wherein the pre-shaped distal sections of the at least two cranial deployment rods lie in the same plane when in an extended deployed state.

7. The vascular closure assembly of claim 5 wherein the pre-shaped distal sections of the at least two caudal deployment rods lie in respective planes disposed at an angle of about 70 degrees to about 110 degrees to each other.

8. The vascular closure assembly of claim 5 wherein a radius of curvature of the pre-shaped distal sections of the cranial deployment rods is about 22 mm to about 30 mm.

9. The vascular closure assembly of claim 5 wherein a radius of curvature of the pre-shaped distal sections of the caudal deployment rods is about 12 mm to about 18 mm.

10. The vascular closure device of claim 1 wherein the deployment rods are configured to translate with respect to the elongate housing but are fixed with respect to rotation about their respective longitudinal axes.

11. The vascular closure assembly of claim 1 wherein each of the deployment rods comprises an anchor engagement section that extends proximally from the distal end of the deployment rod and is angled in a direction opposite that of the curved profile of the pre-shaped distal section.

12. The vascular closure assembly of claim 11 wherein the anchor engagement section of each deployment rod extends proximally from the distal end of the deployment rod by a distance of up to about 0.5 to about 1.5 times an axial length of the anchor.

13. The vascular closure assembly of claim 11 wherein the anchor engagement section of each deployment rod is angled opposite to the curved profile of the pre-shaped distal section by about 16 degrees to about 22 degrees.

14. The vascular closure assembly of claim 5 wherein the pre-shaped distal sections of the cranial deployment rods are configured to include a nominal distal tip angle (without the anchor engagement section) of about 80 degrees to about 90 degrees with respect to a longitudinal axis of the deployment rod which is disposed proximal of the pre-shaped distal section while the pre-shaped distal section is disposed in a relaxed unconstrained state.

15. The vascular closure assembly of claim 5 wherein the pre-curved distal sections of the caudal deployment rods are configured to include a nominal distal tip angle (without the anchor engagement section) of about 110 degrees to about 130 degrees with respect to a longitudinal axis of the deployment rod which is disposed proximal of the pre-shaped distal section while the pre-shaped distal section is disposed in a relaxed unconstrained state.

16. The vascular closure assembly of claim 5 wherein the pre-shaped distal sections of the cranial deployment rods are configured to include a lateral displacement of the distal tip of the deployment rod from the longitudinal axis of the deployment rod normal to the distal tip of about 20 mm to about 30 mm with the pre-shaped distal section of the deployment rod in a relaxed unconstrained state.

17. The vascular closure assembly of claim 5 wherein the pre-shaped distal sections of the caudal deployment rods are configured to include a lateral displacement of the distal tip of the deployment rod from the longitudinal axis of the deployment rod normal to the distal tip of about 20 mm to about 30 mm with the pre-shaped distal section of the deployment rod in a relaxed unconstrained state.

18. The vascular closure assembly of claim 1 wherein the pre-shaped distal section of each of the deployment rods includes a smooth continuous curvature.

19. A vascular closure assembly including an actuator assembly, the actuator assembly including:
a chassis portion;
a plurality of anchor deployers, each anchor deployer including a deployment rod, an anchor which is removably secured to a distal end of the deployment rod, and a filament secured to each anchor; and
an elongate housing, including
a proximal end secured to a distal end of the chassis portion,
a distal end,
an inner lumen extending along the elongate housing to the distal end of the elongate housing,
a plurality of anchor deployer lumens configured to be slidably disposed about a respective anchor deployer, each anchor deployer lumen extending along the elongate housing and terminating distally at a distal port disposed in the distal section of the elongate housing, and
a plurality of filament retainers disposed proximally of the distal ports of the anchor deployer lumens, each filament retainer being configured to releasably secure a section of a respective filament.

20. The vascular closure assembly of claim 19 wherein each of the filament retainers comprise a split tube made from a pliable and resilient material with an inner lumen disposed about a respective filament and a split extending an axial length of the split tube.

21. The vascular closure assembly of claim 20 wherein the split tubes comprise a polymer.

22. The vascular closure device of claim 19 wherein the elongate housing further includes a plurality of anchor pockets, each anchor pocket being disposed adjacent a respective distal port of the anchor deployer lumens and being configured to accept a respective anchor so as to allow a sharpened distal tip of the anchor to be disposed below a nominal outer surface profile of the distal section of the elongate housing while the anchor deployers are in an undeployed state.

23. A vascular closure assembly, comprising:
an inner catheter assembly including an elongate shaft having a proximal end, a distal end, a distal section, an axial length and a guidewire lumen that extends proximally from a distal port at the distal end of the elongate shaft to a proximal port disposed at the distal section; and
an actuator assembly including:
a chassis portion;
a plurality of anchor deployers, each anchor deployer including a deployment rod, an anchor which is removably secured to a distal end of the deployment rod, and a filament secured to each anchor; and
an elongate housing, including
a proximal end secured to a distal end of the chassis portion,
a distal end,
an inner lumen extending along the elongate housing to the distal end of the elongate housing, the inner lumen having an inner surface contour which is configured to be slidably disposed over an outer surface of the elongate shaft,
a plurality of anchor deployer lumens configured to be slidably disposed about a respective anchor deployer, each anchor deployer lumen extending along the elongate housing and terminating distally at a distal port disposed in the distal section of the elongate housing, and
a guidewire relief slot which is disposed in the inner lumen through a wall portion thereof, which extends proximally from the distal end of the inner lumen to a proximal end of the guidewire relief slot, and which is configured to accommodate a guidewire extending outwardly from the proximal port of the guidewire lumen of the elongate shaft.

24. The vascular closure assembly of claim 23 further comprising a guidewire retention clip which extends outwardly from the elongate housing and which is disposed proximally of the proximal end of the guidewire relief slot.

25. A vascular closure assembly, comprising:
an actuator assembly including:
a chassis having a distal end and a proximal end,
a plunger which is proximally translatable with respect to the chassis over a retraction length starting from a distal position,
a tensioner which has a first end secured to the chassis, a second end releasably secured to the plunger and which is configured to continuously apply proximally oriented tension to the plunger with respect to the chassis over the retraction length,
a trigger latch that releasably secures the plunger in the distal position in opposition to the tensioner,
a platen that is distally translatable with respect to the plunger from a proximal cocked position over a deployment length to a distal position that actuates the trigger latch and releases the plunger allowing proximal translation of the plunger over the retraction length,
a compression spring which has a first end operatively coupled to the plunger, a second end operatively coupled to the platen and which is configured to apply a distally oriented force to the platen from the proximal cocked position of the platen over the deployment length to the distal position of the platen,
a platen latch which is operatively coupled to the chassis with a configuration allowing actuation of the platen latch but preventing distal translation of the platen latch with respect to the chassis, which includes a platen catch that is operatively coupled to the platen releasably securing the platen in the proximal cocked position, and
an actuation button which is operatively coupled to the platen latch and configured to actuate the platen latch to disengage the platen catch from the platen;
an elongate housing with a proximal end thereof secured to a distal end of the chassis; and
a plurality of anchor deployers, each anchor deployer being slidably disposed within a respective anchor deployer lumen of the elongate housing and each anchor deployer including
a deployment rod having an elongate resilient configuration which is operatively coupled to the platen such that distal translation of the platen results in distal translation of the deployment rod, and
an anchor which is removably secured to the distal end of the deployment rod.

26. The vascular closure assembly of claim 25 wherein the plunger comprises a tubular configuration that is constrained to translate proximally in an axial direction from the distal position of the plunger and the platen is disposed and translatable within an inner lumen of the tubular plunger.

27. The vascular closure assembly of claim 26 wherein a proximal section of the plunger includes a threaded barrel.

28. The vascular closure assembly of claim 25 wherein the trigger latch comprises a pivoting configuration having a proximal end which is pivotally coupled to the chassis and a distal end which includes a distal facing engagement surface that engages a proximal facing latch surface of the plunger.

29. The vascular closure assembly of claim 25 wherein the tensioner comprises a constant tension spring.

30. The vascular closure assembly of claim 25 wherein the compression spring comprises a helically wound cylindrically shaped spring.

31. A method of actuating an actuator assembly of a vascular closure assembly, comprising
actuating a platen latch of the actuator assembly with an actuation button operatively coupled to a chassis thereby releasing a compression spring, which is operatively coupled between a plunger and a platen, from a compressed state;
translating the platen and deployment rods operatively secured thereto in a distal direction relative to the plunger and chassis under a distal force generated by the released compression spring;
actuating a trigger latch which releasably secures the plunger in a distal position with the platen as it translates distally, thereby releasing the plunger from the fixed distal position;
translating the plunger, platen and deployment rods secured to the platen in a proximal direction under a proximal force generated by a tensioner which is secured to the chassis and releasably secured to the plunger.

32. A vascular closure assembly comprising
a chassis;
an elongate housing having a proximal end secured to a distal end of the chassis;
plurality of anchor deployers which are configured to extend from a distal section of the elongate housing, each anchor deployer including an anchor and a filament secured to the anchor;
a filament lock assembly including
filament tube,
a filament lock having an inner lumen, the inner lumen being disposed over an outer surface of a distal section of the filament tube,
a fairlead having an inner lumen, the inner lumen being disposed over the filament tube axially adjacent the filament lock, and
a polymer bushing disposed between the fairlead and the filament lock.

33. The vascular closure assembly of claim 32 wherein the polymer bushing comprises a polymer.

34. The vascular closure assembly of claim 33 wherein the polymer comprises polyimide.
